SEQUENCE LISTING (19 sequences)

SEQ ID NO: 1 - protein, 403 aa, Homo sapiens [sequence omitted]

SEQ ID NO: 2 - protein, 1210 aa, Homo sapiens [sequence omitted]

SEQ ID NO: 3 - protein, 1225 aa, Homo sapiens
MKLRLPASPETHLDMLRHLYQGCQVVQGNLELTYLPTNASLSFLQDIQEVQGYVLIAHNQVRQVPLQRLRIVRGTQLFEDNYALAVLDNGDPLNNTTPVTGASPGGLRELQLRSLTEILKGGVLIQRNPQLCYQDTILWKDIFHKNNQLALTLIDTNRSRACHPCSPMCKGSRCWGESSEDCQSLTRTVCAGGCARCKGPLPTDCCHEQCAAGCTGPKHSDCLACLHFNHSGICELHCPALVTYNTDTFESMPNPEGRYTFGASCVTACPYNYLSTDVGSCTLVCPLHNQEVTAEDGTQRCEKCSKPCARVCYGLGMEHLREVRAVTSANIQEFAGCKKIFGSLAFLPESFDGDPASNTAPLQPEQLQVFETLEEITGYLYISAWPDSLPDLSVFQNLQVIRGRILHNGAYSLTLQGLGISWLGLRSLRELGSGLALIHHNTHLCFVHTVPWDQLFRNPHQALLHTANRPEDECVGEGLACHQLCARGHCWGPGPTQCVNCSQFLRGQECVEECRVLQGLPREYVNARHCLPCHPECQPQNGSVTCFGPEADQCVACAHYKDPPFCVARCPSGVKPDLSYMPIWKFPDEEGACQPCPINCTHSCVDLDDKGCPAEQRASPLTSIISAVVGILLVVVLGVVFGILIKRRQQKIRKYTMRRLLQETELVEPLTPSGAMPNQAQMRILKETELRKVKVLGSGAFGTVYKGIWIPDGENVKIPVAIKVLRENTSPKANKEILDEAYVMAGVGSPYVSRLLGICLTSTVQLVTQLMPYGCLLDHVRENRGRLGSQDLLNWCMQIAKGMSYLEDVRLVHRDLAARNVLVKSPNHVKITDFGLARLLDIDETEYHADGGKVPIKWMALESILRRRFTHQSDVWSYGVTVWELMTFGAKPYDGIPAREIPDLLEKGERLPQPPICTIDVYMIMVKCWMIDSECRPRFRELVSEFSRMARDPQRFVVIQNEDLGPASPLDSTFYRSLLEDDDMGDLVDAEEYLVPQQGFFCPDPAPGAGGMVHHRHRSSSTRSGGGDLTLGLEPSEEEAPRSPLAPSEGAGSDVFDGDLGMGAAKGLQSLPTHDPSPLQRYSEDPTVPLPSETDGYVAPLTCSPQPEYVNQPDVRPQPPSPREGPLPAARPAGATLERPKTLSPGKNGVVKDVFAFGGAVENPEYLTPQGGAAPQPHPPPAFSPAFDNLYYWDQDPPERGAPPSTFKGTPTAENPEYLGLDVPV

SEQ ID NO: 4 - protein, 1210 aa, Homo sapiens [sequence omitted]

SEQ ID NO: 5 - protein, 249 aa, Homo sapiens
MKLNISFPATGCQKLIEVDDERKLRTFYEKRMATEVAADALGEEWKGYVVRISGGNDKQGFPMKQGVLTHGRVRLLLSKGHSCYRPRRTGERKRKSVRGCIVDANLSVLNLVIVKKGEKDIPGLTDTTVPRRLGPKRASRIRKLFNLSKEDDVRQYVVRKPLNKEGKKPRTKAPKIQRLVTPRVLQHKRRRIALKKQRTKKNKEEAAEYAKLLAKRMKEAKEKRQEQIAKRRRLSSLRASTSKSESSQK

SEQ ID NO: 6 - protein, 480 aa, Homo sapiens
MSDVAIVKEGWLHKRGEYIKTWRPRYFLLKNDGTFIGYKERPQDVDQREAPLNNFSVAQCQLMKTERPRPNTFIIRCLQWTTVIERTFHVETPEEREEWTTAIQTVADGLKKQEEEEMDFRSGSPSDNSGAEEMEVSLAKPKHRVTMNEFEYLKLLGKGTFGKVILVKEKATGRYYAMKILKKEVIVAKDEVAHTLTENRVLQNSRHPFLTALKYSFQTHDRLCFVMEYANGGELFFHLSRERVFSEDRARFYGAEIVSALDYLHSEKNVVYRDLKLENLMLDKDGHIKITDFGLCKEGIKDGATMKTFCGTPEYLAPEVLEDNDYGRAVDWWGLGVVMYEMMCGRLPFYNQDHEKLFELILMEEIRFPRTLGPEAKSLLSGLLKKDPKQRLGGGSEDAKEIMQHRFFAGIVWQHVYEKKLSPPFKPQVTSETDTRYFDEEFTAQMITITPPDQDDSMECVDSERRPHFPQFSYSASSTA

SEQ ID NO: 7 - protein, 379 aa, Homo sapiens
MAAAAAQGGGGGEPRRTEGVGPGVPGEVEMVKGQPFDVGPRYTQLQYIGEGAYGMVSSAYDHVRKTRVAIKKISPFEHQTYCQRTLREIQILLRFRHENVIGIRDILRASTLEAMRDVYIVQDLMETDLYKLLKSQQLSNDHICYFLYQILRGLKYIHSANVLHRDLKPSNLLINTTCDLKICDFGLARIADPEHDHTGFLTEYVATRWYRAPEIMLNSKGYTKSIDIWSVGCILAEMLSNRPIFPGKHYLDQLNHILGILGSPSQEDLNCIINMKARNYLQSLPSKTKVAWAKLFPKSDSKALDLLDRMLTFNPNKRITVEEALAHPYLEQYYDPTDEPVAEEPFTFAMELDDLPKERLKELIFQETARFQPGVLEAP

SEQ ID NO: 8 - DNA, 3413 bp, Homo sapiens
cctcccctcgcccggcgcggtcccgtccgcctctcgctcgcctcccgcctcccctcggtcttccgaggcgcccgggctcccggcgcggcggcggagggggcgggcaggccggcgggcggtgatgtggcgggactctttatgcgctgcggcaggatacgcgctcggcgctgggacgcgactgcgctcagttctctcctctcggaagctgcagccatgatggaagtttgagagttgagccgctgtgaggcgaggccgggctcaggcgagggagatgagagacggcggcggccgcggcccggagcccctctcagcgcctgtgagcagccgcgggggcagcgccctcggggagccggccggcctgcggcggcggcagcggcggcgtttctcgcctcctcttcgtcttttctaaccgtgcagcctcttcctcggcttctcctgaaagggaaggtggaagccgtgggctcgggcgggagccggctgaggcgcggcggcggcggcggcacctcccgctcctggagcgggggggagaagcggcggcggcggcggccgcggcggctgcagctccagggagggggtctgagtcgcctgtcaccatttccagggctgggaacgccggagagttggtctctccccttctactgcctccaacacggcggcggcggcggcggcacatccagggacccgggccggttttaaacctcccgtccgccgccgccgcaccccccgtggcccgggctccggaggccgccggcggaggcagccgttcggaggattattcgtcttctccccattccgctgccgccgctgccaggcctctggctgctgaggagaagcaggcccagtcgctgcaaccatccagcagccgccgcagcagccattacccggctgcggtccagagccaagcggcggcagagcgaggggcatcagctaccgccaagtccagagccatttccatcctgcagaagaagccccgccaccagcagcttctgccatctctctcctcctttttcttcagccacaggctcccagacatgacagccatcatcaaagagatcgttagcagaaacaaaaggagatatcaagaggatggattcgacttagacttgacctatatttatccaaacattattgctatgggatttcctgcagaaagacttgaaggcgtatacaggaacaatattgatgatgtagtaaggtttttggattcaaagcataaaaaccattacaagatatacaatctttgtgctgaaagacattatgacaccgccaaatttaattgcagagttgcacaatatccttttgaagaccataacccaccacagctagaacttatcaaacccttttgtgaagatcttgaccaatggctaagtgaagatgacaatcatgttgcagcaattcactgtaaagctggaaagggacgaactggtgtaatgatatgtgcatatttattacatcggggcaaatttttaaaggcacaagaggccctagatttctatggggaagtaaggaccagagacaaaaagggagtaactattcccagtcagaggcgctatgtgtattattatagctacctgttaaagaatcatctggattatagaccagtggcactgttgtttcacaagatgatgtttgaaactattccaatgttcagtggcggaacttatcctcagtttgtggtctgccagctaaaggtgaagatatattcctccaattcaggacccacacgacgggaagacaagttcatgtactttgagttccctcagccgttacctgtgtgtggtgatatcaaagtagagttcttccacaaacagaacaagatgctaaaaaaggacaaaatgtttcacttttgggtaaatacattcttcataccaggaccagaggaaacctcagaaaaagtagaaaatggaagtctatgtgatcaagaaatcgatagcatttgcagtatagagcgtgcagataatgacaaggaatatctagtacttactttaacaaaaaatgatcttgacaaagcaaataaagacaaagccaaccgatacttttctccaaattttaaggtgaagctgtacttcacaaaaacagtagaggagccgtcaaatccagaggctagcagttcaacttctgtaacaccagatgttagtgacaatgaacctgatcattatagatattctgacaccactgactctgatccagagaatgaaccttttgatgaagatcagcatacacaaattacaaaagtctgaatttttttttatcaagagggataaaacaccatgaaaataaacttgaataaactgaaaatggacctttttttttttaatggcaataggacattgtgtcagattaccagttataggaacaattctcttttcctgaccaatcttgttttaccctatacatccacagggttttgacacttgttgtccagttgaaaaaaggttgtgtagctgtgtcatgtatatacctttttgtgtcaaaaggacatttaaaattcaattaggattaataaagatggcactttcccgttttattccagttttataaaaagtggagacagactgatgtgtatacgtaggaattttttccttttgtgttctgtcaccaactgaagtggctaaagagctttgtgatatactggttcacatcctacccctttgcacttgtggcaacagataagtttgcagttggctaagagaggtttccgaagggttttgctacattctaatgcatgtattcgggttaggggaatggagggaatgctcagaaaggaaataattttatgctggactctggaccatataccatctccagctatttacacacacctttctttagcatgctacagttattaatctggacattcgaggaattggccgctgtcactgcttgttgtttgcgcatttttttttaaagcatattggtgctagaaaaggcagctaaaggaagtgaatctgtattggggtacaggaatgaaccttctgcaacatcttaagatccacaaatgaagggatataaaaataatgtcataggtaagaaacacagcaacaatgacttaaccatataaatgtggaggctatcaacaaagaatgggcttgaaacattataaaaattgacaatgatttattaaatatgttttctcaattgtaacgacttctccatctcctgtgtaatcaaggccagtgctaaaattcagatgctgttagtacctacatcagtcaacaacttacacttattttactagttttcaatcataatacctgctgtggatgcttcatgtgctgcctgcaagcttcttttttctcattaaatataaaatattttgtaatgctgcacagaaattttcaatttgagattctacagtaagcgttttttttctttgaagatttatgatgcacttattcaatagctgtcagccg

SEQ ID NO: 9 - DNA, 1595 bp, Homo sapiens
ccccggcgcagcgcggccgcagcagcctccgccccccgcacggtgtgagcgcccgacgcggccgaggcggccggagtcccgagctagccccggcggccgccgccgcccagaccggacgacaggccacctcgtcggcgtccgcccgagtccccgcctcgccgccaacgccacaaccaccgcgcacggccccctgactccgtccagtattgatcgggagagccggagcgagctcttcggggagcagcgatgcgaccctccgggacggccggggcagcgctcctggcgctgctggctgcgctctgcccggcgagtcgggctctggaggaaaagaaagtttgccaaggcacgagtaacaagctcacgcagttgggcacttttgaagatcattttctcagcctccagaggatgttcaataactgtgaggtggtccttgggaatttggaaattacctatgtgcagaggaattatgatctttccttcttaaagaccatccaggaggtggctggttatgtcctcattgccctcaacacagtggagcgaattcctttggaaaacctgcagatcatcagaggaaatatgtactacgaaaattcctatgccttagcagtcttatctaactatgatgcaaataaaaccggactgaaggagctgcccatgagaaatttacaggaaatcctgcatggcgccgtgcggttcagcaacaaccctgccctgtgcaacgtggagagcatccagtggcgggacatagtcagcagtgactttctcagcaacatgtcgatggacttccagaaccacctgggcagctgccaaaagtgtgatccaagctgtcccaatgggagctgctggggtgcaggagaggagaactgccagaaactgaccaaaatcatctgtgcccagcagtgctccgggcgctgccgtggcaagtcccccagtgactgctgccacaaccagtgtgctgcaggctgcacaggcccccgggagagcgactgcctggtctgccgcaaattccgagacgaagccacgtgcaaggacacctgccccccactcatgctctacaaccccaccacgtaccagatggatgtgaaccccgagggcaaatacagctttggtgccacctgcgtgaagaagtgtccccgtaattatgtggtgacagatcacggctcgtgcgtccgagcctgtggggccgacagctatgagatggaggaagacggcgtccgcaagtgtaagaagtgcgaagggccttgccgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacaggtttgagctgaattatcacatgaatataaatgggaaatcagtgttttagagagagaacttttcgacatatttcctgttcccttggaataaaaacatttcttctgaaattttaccgttaaaaaaaaaaaaaaaaaaaaaaaaa

SEQ ID NO: 10 - DNA, 22 bp, Artificial Sequence
  Description of Artificial Sequence Synthetic PCR primer
accaatacctattccgttacac

SEQ ID NO: 11 - DNA, 22 bp, Artificial Sequence
  Description of Artificial Sequence Synthetic PCR primer
tgatgacaagcttcccgttctc

SEQ ID NO: 12 - DNA, 20 bp, Artificial Sequence
  Description of Artificial Sequence Synthetic PCR primer
aaagagtgctcaccgcagtt

SEQ ID NO: 13 - DNA, 20 bp, Artificial Sequence
  Description of Artificial Sequence Synthetic PCR primer
cactggatgctctccacgtt

SEQ ID NO: 14 - DNA, 20 bp, Artificial Sequence
  Description of Artificial Sequence Synthetic PCR primer
cttcaaaaactgcacctcca

SEQ ID NO: 15 - DNA, 21 bp, Artificial Sequence
  Description of Artificial Sequence Synthetic PCR primer
caagcaactgaacctgtgact

SEQ ID NO: 16 - DNA, 20 bp, Artificial Sequence
  Description of Artificial Sequence Synthetic PCR primer
cagcaagagcacaagaggaa

SEQ ID NO: 17 - DNA, 20 bp, Artificial Sequence
  Description of Artificial Sequence Synthetic PCR primer
caactgtgaggaggggagat

SEQ ID NO: 18 - DNA, 22 bp, Artificial Sequence
  Description of Artificial Sequence Synthetic PCR primer
cttcggggagcagcgatgcgac

SEQ ID NO: 19 - DNA, 20 bp, Artificial Sequence
  Description of Artificial Sequence Synthetic PCR primer
gtgaaggtcggagtcaacgg

(19) United States
(12) Patent Application Publication
(10) Pub. No.: US 2011/0045459 A1
(43) Pub. Date: Feb. 24, 2011

(54) Molecular determinants of EGFR kinase inhibitor response in glioblastoma

(76) Inventors: Paul S. Mischel, Los Angeles, CA (US); Ingo K. Mellinghoff, Los Angeles, CA (US); Yinglin Wang, Los Angeles, CA (US); Timothy F. Cloughesy, Encino, CA (US); Charles L. Sawyers, Los Angeles, CA (US)

Correspondence Address: GATES & COOPER LLP;HOWARD HUGHES CENTER, 6701 CENTER DRIVE WEST, SUITE 1050, LOS ANGELES, CA, 90045, US

(21) Appl. No.: 11/918,916

(22) PCT Filed: Apr. 21, 2006
(86) PCT No.: PCT/US2006/014981

§ 371 Date: Oct. 19, 2007

Related U.S. Application Data
(60) Provisional application No. 60/673,607, filed on Apr. 21, 2005.

(51) Int. Cl. C12Q 1/68; G01N 33/574; C12Q 1/42

(52) U.S. Cl. 435/6; 435/7.23; 435/21

(57) ABSTRACT

The invention disclosed herein provides methods for the examination and/or quantification of biochemical pathways that are disregulated in pathologies such as cancer and to reagents and kits adapted for performing such methods.

### CROSS REFERENCE TO RELATED APPLICATIONS

This application claims the benefit of U.S. provisional patent application Ser. No. 60/673,607 filed Apr. 21, 2005, the contents of which are incorporated by reference. This application is also related to International Application Number PCT/US2004/037288 which is a continuation-in-part of U.S. patent application Ser. No. 10/701,490 filed Nov. 5, 2003, which claims the benefit of U.S. Provisional Application Ser. No. 60/423,777 filed Nov. 5, 2002, the contents of each of which are incorporated herein by reference. This application is also related to U.S. Provisional Application Ser. No. 60/662,649 filed Mar. 17, 2005, the contents of which are incorporated herein by reference.

### STATEMENT OF GOVERNMENT SUPPORT

This invention was made with Govermnent support from RO1 NS50151 from the National Institutes of Health. The Government may have certain rights to this invention.

### FIELD OF THE INVENTION

The present invention provides methods for the examination of pathologies such as cancer and to reagents adapted for performing these methods.

### BACKGROUND OF THE INVENTION

Cancers are the second most prevalent cause of death in the United States, causing 450,000 deaths per year. One in three Americans will develop cancer, and one in five will die of cancer. While substantial progress has been made in identifying some of the likely environmental and hereditary causes of cancer, there is a need for additional diagnostic and therapeutic modalities that target cancer and related diseases and disorders. In particular, there is for a need a greater understanding of the various biochemical pathways that are involved in disregulated cell growth such as cancer as this will allow for the development of improved diagnostic and therapeutic methods for identifying and treating pathological syndromes associated with such growth disregulation.

Tyrosine kinases are key regulators of intracellular signaling (see, e.g. Pawson et al., Science 2003300:445-52; and Manning et al., Sci STKF, 2002; 2002:PE49). Over-expressed or mutated tyrosine kinases contribute to the development and progression of tumors, and are found in many types of cancer (see, e.g. Sawyers C L. Genes Dev 2003; 17:2998-3010; van Oosterom et al., Lancet 2001; 358:1421-3; and Demetri G D et al., N Engl J Med 2002; 347:472-80). The enhanced dependence of tumor cells on chronically activated tyrosine kinases may potentially render patients responsive to targeted kinase inhibitor therapy (see, e.g., Sawyers C L. Genes Dev 2003; 17:2998-3010; van Oosterom et al., Lancet 2001; 358:1421-3; Demetri G D et al., N Engl J Med 2002; 347:472-80; Druker B J et al., N Engl J Med 2001; 344:1038-42; and Druker B J et al., N Engl J Med 2001; 344:1031-7). EGFR (SEQ ID: 4), a receptor tyrosine kinase that is amplified and/or mutated in a number of neoplasms, is thought to be a potentially important therapeutic target (see, e.g., Dancey J E et al., Lancet 2003; 362:62-4). Among lung cancer patients, a relatively small subset respond to EGFR inhibitors (see, e.g., Kris M G et al., Jama 2003; 290:2149-58; Fukuoka M et al., J Clin Oncol 2003; 21:2237-46; and Miller V A et al., J Clin Oncol 2004; 22:1103-9), and EGFR kinase domain mutations are significantly associated with response (see, e.g., Lynch T J et al., N Engl J Med 2004; 350:2129-39; Paez J G et al., Science 2004; 304:1497-500; and Pao W et al., Proc Natl Acad Sci USA 2004; 101:13306-11). It is not yet known whether EGFR kinase domain mutations are important for determining response in other types of cancer.

Glioblastoma, the most common malignant primary brain tumor of adults has also been targeted with EGFR inhibitor therapy. Unlike in lung cancer, large phase II clinical trials of EGFR inhibitors in glioblastoma have not been performed. However it is clear that a small subset of glioblastoma patients derive benefit from this class of agents (see e.g., Prados M et al., Proceedings of the American Society of Clinical Oncology 2003; Abstract 394 and Rich J N et al., J Clin Oncol 2004; 22:133-42). EGFR kinase domain mutations were not detected in nine glioblastoma patients who had prolonged survival on gefitinib following surgical resection, raising the possibility of a different mechanism of sensitization to EGFR inhibitors (see e.g., Rich J N et al., N Engl J Med 2004; 351:1260-1; author reply 1260-1). The EGFR gene is commonly amplified in glioblastoma (see e.g., Smith J S et al., J Natl Cancer Inst 2001; 93:1246-56), but this also does not appear to correlate with response to EGFR inhibitors (see, e.g., Rich J N et al., J Clin Oncol 2004; 22:133-42). Identifying the molecular mechanism underlying clinical response is critical for application of this therapy to glioblastoma patients.

### SUMMARY OF THE INVENTION

The epidermal growth factor receptor (EGFR) is frequently amplified, overexpressed or mutated in glioblastomas, but only 10-20% of patients respond to EGFR kinase inhibitors. EGFR kinase domain mutations are strongly associated with clinical response to EGFR inhibitors in lung cancer, but glioblastomas may use an alternate mechanism of sensitization. Glioblastomas commonly express EGFRvIII, a chronically active genomic deletion variant of EGFR (see, e.g., Aldape K D et al., J Neuropathol Exp Neurol 2004; 63:700-7; Frederick L et al., Cancer Res 2000; 60:1383-7; Wong A J et al., Proc Natl Acad Sci USA 1992; 89:2965-9; Sugawa N et al., Proc Natl. Acad Sci USA 1990; 87:8602-6; and Ekstrand A J et al., Cancer Res 1991; 51:2164-72). Like some of the reported EGFR kinase domain mutations, EGFRvIII has enhanced activity relative to wild type EGFR and strongly promotes PI3K pathway signaling (see e.g., Sordella R et al., Science 2004; 305:1163-7; Choe G et al., Cancer Res 2003; 63:2742-6; Li B et al., Oncogene 2004; 23:4594-602; Huang H S, et al., J Biol Chem 1997; 272:2927-35; and Batra S K et al., Cell Growth Differ 1995; 6:1251-9). Thus, we reasoned that similar to EGFR kinase domain mutations in lung cancer, EGFRvIII may sensitize glioblastoma patients to EGFR kinase inhibitors.

Glioblastomas also commonly lose expression of the PTEN tumor suppressor protein (see e.g., Smith J S et al., J Natl. Cancer Inst 2001; 93:1246-56; Choe G et al., Cancer Res 2003; 63:2742-6; and Ermoian R P et al., Clin Cancer Res 2002; 8:1100-6). This results in chronic PI3K pathway activation (see. e.g., Choe G et al., Cancer Res 2003; 63:2742-6; and Ermoian R P et al., Clin Cancer Res 2002; 8:1100-6) and impairs response to EGFR family kinase inhibitors (see, e.g., Bianco R et al., Oncogene 2003; 22:2812-22; and Nagata Y et al., Cancer Cell 2004; 6:117-27). Thus, we hypothesized that PTEN loss could potentially render glioblastomas insensitive to EGFR kinase inhibitors, even when EGFRvIII is expressed. To test these hypotheses, we analyzed tumor tissue from glioblastoma patients treated with EGFR kinase inhibitors. We searched for mutations in the EGFR and Her2/Neu genes analyzed EGFR, EGFRvIII and PTEN at the gene and protein level, determined the molecular correlates of response, confirmed them in an independent dataset, and validated our findings in a cell culture model.

We searched for kinase domain mutations in EGFR and human epidermal growth factor receptor type 2 (Her2/Neu) and analyzed gene and protein expression of EGFR, EGFR deletion mutant variant III (EGFRvIII) and phosphatase and tensin homologue deleted on chromosome 10 (PTEN) in recurrent malignant glioma patients treated with EGFR kinase inhibitors. We determined molecular correlates of clinical response, confirmed them in an independent dataset, and validated them in vitro.

Forty-nine patients with recurrent malignant glioma were treated at UCLA with EGFR inhibitors and 9/49 (18%) demonstrated >25% tumor shrinkage. Pre-treatment tissue was available for molecular analysis from 26 patients, seven who responded and nineteen who rapidly progressed on therapy. EGFR and Her2/Neu kinase domain mutations were not detected in any EGFR inhibitor treated patients. EGFRvIII/PTEN protein coexpression was significantly associated with clinical response (p=0.00078; O.R.=51.0; 95% CI=(3.89-669)). These findings were validated in a dataset of 33 patients treated at a different institution. In vitro, EGFRvIII sensitized glioblastoma cells to erlotinib, but only when PTEN was coexpressed.

In summary, in glioblastoma, EGFRvIII and PTEN coexpression correlates with clinical response to EGFR kinase inhibitors while EGFR and Her2/Neu kinase domain mutations do not play a major role in determining patient response. Consequently, screening for EGFRvIII and PTEN may help identify patients most likely to respond to EGFR kinase inhibitor therapy. In this context, the invention disclosed herein has a number of embodiments. A first embodiment is a method for identifying a mammalian tumor cell that is likely to respond, or is responsive to an epidermal growth factor receptor (EGFR) inhibitor (e.g. erlotinib or gefitinib), the method comprising examining mammalian tumor cell for the expression of the EGFR deletion mutant variant III (EGFRvIII) mRNA or protein and the expression of the phosphatase and tensity homologue deleted on chromosome 10 (PTEN) mRNA or protein, wherein the coexpression of EGFRvIII and PTEN mRNA or protein identifies the mammalian tumor cell as likely to respond or responsive to an epidermal growth factor receptor (EGFR) inhibitor. Typically in these methods, the mammalian tumor cell is a glioma such as glioblastoma.

Optionally in such methods, the coexpression of EGFRvIII and PTEN proteins is examined using an antibody that binds EGFRvIII protein and an antibody that binds PTEN protein, for example in immunohistochemistry or immunoblotting protocols. Alternatively, the coexpression of EGFRvIII and PTEN mRNA in the cell are evaluated by contacting the cell with EGFRvIII and PTEN complementary polynucleotides that hybridize to EGFRvIII and PTEN mRNAs, for example in Northern analysis or polymerase chain reaction analysis protocols.

The invention also provides additional articles of manufacture including kits. In one such embodiment of the invention, a kit having a reagent useful for sensing EGFRvIII and PTEN mRNA or protein, is provided. The kit typically comprises a container, a label and a EGFRvIII and/or PTEN probe, primer or antibody.

### BRIEF DESCRIPTION OF THE DRAWINGS

FIG. 1. A subset of recurrent malignant glioma patients respond to EGFR kinase inhibitors. A. Representative MRI images to assess response to EGFR kinase inhibitors. Patient 3 had significant tumor shrinkage relative to baseline in this scan taken 8 weeks after starting EGFR inhibitor therapy. B. Patient 22 had considerable tumor growth relative to baseline in this scan taken after 7 weeks of therapy. C. Median time to progression (TTP) (+/−interquartile range) for recurrent malignant glioma patients treated with erlotinib or gefitinib, stratified by MRI response. Responders demonstrated >25% decrease in bi-directional tumor area; non-responders with progressive disease had >25% tumor growth while on EGFR kinase inhibitors. Patients classified as responders by MRI criteria had significantly prolonged time to progression relative to non-responder patients (p=0.00029). The two anaplastic oligodendroglioma patients (1 responder, 1 non-responder) were not included in time to progression analysis, since this tumor is generally associated with less rapid progression than glioblastoma.

FIG. 2. Detection of the EGFRvIII and PTEN in patient samples. A. Detection of EGFRvIII in fresh frozen tumor samples by RT-PCR and immunoblotting. Upper panel: RT-PCR based detection of EGFRvIII. Primers flanking the exon 2-7 deletion in EGFRvIII amplify cDNA fragments from both full-length EGFR (1043 basepairs) and the truncated EGFRvIII (252 basepairs). Plasmid cDNAs for wildtype EGFR (lane 1) and EGFRvIII (lane 2) were included as size controls. Lower panel: Immunoblotting of GBM tumor lysates with a panreactive EGFR antibody which detects both full length EGFR (˜170 kDa) and the truncated EGFRvIII (˜140 kDa). Whole cell lysates from U87-EGFR (lane 1) and U87-EGFRvIII (lane 2) cell lines were included as controls. B. The EGFR antibody L8A4 (see, e.g., Wikstrand C J et al., Cancer Res 1997; 57:4130-40) (a gift from Dr. Darell Bigner) reacts with EGFRvIII, but not with full length EGFR. Whole cell lysates from three isogenic U87 GBM sublines (U87, U87-EGFR, and U87-EGFRvIII) were subjected to SDS-PAGE and probed with L8A4 (top panel), a pan-reactive EGFR/EGFRvIII antibody (middle panel), and anti-tubulin as loading control. C. Immunohistochemical detection and quantification of EGFRvIII status in paraffin-embedded tumor samples. Tumor samples and adjacent normal brain tissue were stained with L8A4. Inset shows a higher magnification view and staining of the cytoplasmic membrane. Representative “false-color” images were generated using the Soft-Imaging Systems image analysis software. An EGFRvIII “IHC-score” was calculated by dividing the mean red-brown color saturation per cell in tumor tissue by the mean red-brown color saturation per cell in adjacent normal brain tissue. The graph shows the correlation between the EGFRvIII IHC-score and non-IHC based EGFRVIII detection methods (RT-PCR, Immunoblotting, EGFR exon9/exon4-ratio by quantitative PCR). D. Detection of PTEN in patient samples. Representative immunohistochemical staining (upper panel) and immunoblotting (lower panel) from a PTEN deficient tumor (patient 16) and a PTEN intact tumor (patient 23) are shown. Note the loss of PTEN staining in tumor cells, but not in vascular endothelial cells in sample 16. PTEN immunoblotting of whole cell lysates from isogenic U87 cells with or without PTEN over-expression, demonstrate the specificity of the antibody.

FIG. 3. EGFRvIII and PTEN co-expression sensitize glioblastoma cells to erlotinib. A. Immunoblot blot analysis of EGFR, EGFRvIII and PTEN expressing stable cell lines showing downstream signaling effects of their expression. B. EGFRvIII and PTEN co-expression sensitizes glioblastoma cells to erlotinib. 1000 cells/well of each cell line was seeded into 96-well plates in 8 replicates. Erlotinib was added to wells after 24 hours with final concentrations ranging from 0-10 μM. Plates were incubated for 10-14 days, fixed, stained with crystal violet and quantified. Experiments were repeated 3 times in 8 replicates for each condition.

### DETAILED DESCRIPTION OF THE INVENTION

The techniques and procedures described or referenced herein are generally well understood and commonly employed using conventional methodology by those skilled in the art, such as, for example, the widely utilized molecular cloning methodologies described in Ausubel et al., Current Protocols in Molecular Biology, Wiley Interscience Publishers, (1995). As appropriate, procedures involving the use of commercially available kits and reagents are generally carried out in accordance with manufacturer defined protocols and/or parameters unless otherwise noted. Unless otherwise defined, all terms of art, notations and other scientific terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a substantial difference over what is generally understood in the art.

“Mammal” for purposes of treatment or therapy refers to any animal classified as a mammal, including humans, domestic and farm animals, and zoo, sports, or pet animals, such as dogs, horses, cats, cows, etc. Preferably, the mammal is human.

By “subject” or “patient” is meant any single subject for which therapy is desired, including humans, cattle, dogs, guinea pigs, rabbits, chickens, insects and so on. Also intended to be included as a subject are any subjects involved in clinical research trials not showing any clinical sign of disease, or subjects involved in epidemiological studies, or subjects used as controls.

The terms “cancer”, “cancerous”, or “malignant” refer to or describe the physiological condition in mammals that is typically characterized by unregulated growth of mammalian tumor cells. Examples of cancer include but are not limited to astrocytoma, blastoma, carcinoma, glioblastoma, leukemia, lymphoma and sarcoma. More particular examples of such cancers include adrenal, and ophthalmologic cancers, brain cancer breast cancer, ovarian cancer, colon cancer, colorectal cancer, rectal cancer, squamous cell cancer, small-cell lung cancer, non-small cell lung cancer, Hodgkin's and non-Hodgkin's lymphoma, testicular cancer, esophageal cancer, gastrointestinal cancer, renal cancer, pancreatic cancer, glioblastoma, cervical cancer, glioma, liver cancer, bladder cancer, hepatoma, endometrial carcinoma, salivary gland carcinoma, kidney cancer, liver cancer, prostate cancer, vulval cancer, thyroid cancer, hepatic carcinoma and various types of head and neck cancer.

“Growth inhibition” when used herein refers to the growth inhibition of a cell in vitro and/or in vivo. The inhibition of cell growth can be measured by a wide variety of methods known in the art. A “growth inhibitory agent” when used herein refers to a compound or composition which inhibits growth of a cell in vitro and/or in vivo. Thus, the growth inhibitory agent may be one which significantly reduces the percentage of cells in S phase. Examples of growth inhibitory agents include agents that block cell cycle progression (at a place other than S phase), such as agents that induce G1 arrest and M-phase arrest. Classical M-phase blockers include the vincas (vincristine and vinblastine), TAXOL®, and topo II inhibitors such as doxorubicin, epirubicin, daunorubicin, etoposide, and bleomycin. Those agents that arrest G1 also spill over into S-phase arrest, for example, DNA alkylating agents such as tamoxifen, prednisone, dacarbazine, mechlorethamine, cisplatin, methotrexate, 5-fluorouracil, and ara-C. Such agents further include inhibitors of cellular pathways associated with disregulated cell growth such as the PI3K/Akt pathway. Further information can be found in The Molecular Basis of Cancer, Mendelsohn and Israel, eds., Chapter 1, entitled “Cell cycle regulation, oncogenes, and antineoplastic drugs” by Murakami et al. (W B Saunders: Philadelphia, 1995).

“Treatment” or “therapy” refer to both therapeutic treatment and prophylactic or preventative measures. The term “therapeutically effective amount” refers to an amount of a drug effective to treat a disease or disorder in a mammal. In the case of cancer, the therapeutically effective amount of the drug may reduce the number of cancer cells; reduce the tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the disorder. To the extent the drug may prevent growth and/or kill existing cancer cells, it may be cytostatic and/or cytotoxic. For cancer therapy, efficacy in vivo can, for example, be measured by assessing tumor burden or volume, the time to disease progression (TTP) and/or determining the response rates (RR).

By “tissue sample” is meant a collection of similar cells obtained from a tissue of a subject or patient, preferably containing nucleated cells with chromosomal material. The four main human tissues are (1) epithelium; (2) the connective tissues, including blood vessels, bone and cartilage; (3) muscle tissue; and (4) nerve tissue. The source of the tissue sample may be solid tissue as from a fresh, frozen and/or preserved organ or tissue sample or biopsy or aspirate; blood or any blood constituents; bodily fluids such as cerebral spinal fluid, amniotic fluid, peritoneal fluid, or interstitial fluid; cells from any time in gestation or development of the subject. The tissue sample may also be primary or cultured cells or cell lines. The tissue sample may contain compounds which are not naturally intermixed with the tissue in nature such as preservatives, anticoagulants, buffers, fixatives, nutrients, antibiotics, or the like. In one embodiment of the invention, the tissue sample is “non-hematologic tissue” (i.e. not blood or bone marrow tissue).

For the purposes herein a “section” of a tissue sample is meant a single part or piece of a tissue sample, e.g. a thin slice of tissue or cells cut from a tissue sample. It is understood that multiple sections of tissue samples may be taken and subjected to analysis according to the present invention, provided that it is understood that the present invention comprises a method whereby the same section of tissue sample is analyzed at both morphological and molecular levels, or is analyzed with respect to both protein and nucleic acid.

“Stringency” of hybridization reactions is readily determinable by one of ordinary skill in the art, and generally is an empirical calculation dependent upon probe length, washing temperature, and salt concentration. In general, longer probes require higher temperatures for proper annealing, while shorter probes need lower temperatures. Hybridization generally depends on the ability of denatured DNA to re-anneal when complementary strands are present in an environment below their melting temperature. The higher the degree of desired identity between the probe and hybridizable sequence, the higher the relative temperature which can be used. As a result, it follows that higher relative temperatures would tend to make the reaction conditions more stringent, while lower temperatures less so. For additional details and explanation of stringency of hybridization reactions, see Ausubel et al., Current Protocols in Molecular Biology, Wiley Interscience Publishers, (1995).

“High stringency conditions”, as defined herein, are identified by those that: (1) employ low ionic strength and high temperature for washing; 0.015 M sodium chloride/0.0015 M sodium citrate/0.1% sodium dodecyl sulfate at 50° C.; (2) employ during hybridization a denaturing agent; 50% (v/v) formamide with 0.1% bovine serum albumin/0.1% Ficoll/0.1% polyvinylpyrrolidone/50 mM sodium phosphate buffer at pH 6.5 with 750 mM sodium chloride, 75 mM sodium citrate at 42° C.; or (3) employ 50% formamide, 5×SSC (0.75 M NaCl, 0.075 M sodium citrate), 50 mM sodium phosphate (pH 6.8), 0.1% sodium pyrophosphate, 5× Denhardt's solution, sonicated salmon sperm DNA (50 μg/ml), 0.1% SDS, and 10% dextran sulfate at 42° C., with washes at 42° C. in 0.2×SSC (sodium chloride/sodium citrate) and 50% formamide at 55° C., followed by a high-stringency wash consisting of 0.1×SSC containing EDTA at 55° C.

“Moderately stringent conditions” may be identified as described by Sambrook et al., Molecular Cloning: A Laboratory Manual, New York: Cold Spring Harbor Press, 1989, and include overnight incubation at 37° C. in a solution comprising: 20% formamide, 5×SSC (150 mM NaCl, 15 mM trisodium citrate), 50 mM sodium phosphate (pH 7.6), 5× Denhardt's solution, 10% dextran sulfate, and 20 mg/ml denatured sheared salmon sperm DNA, followed by washing the filters in 1×SSC at about 37-50° C. The skilled artisan will recognize how to adjust the temperature, ionic strength, etc. as necessary to accommodate factors such as probe length and the like.

By “correlate” or “correlating” is meant comparing, in any way, the performance and/or results of a first analysis or protocol with the performance and/or results of a second analysis or protocol. For example, one may use the results of a first analysis or protocol in carrying out a second protocols and/or one may use the results of a first analysis or protocol to determine whether a second analysis or protocol should be performed. With respect to the embodiment of immununohistochemical analysis or protocol one may use the results of IHC to determine whether a specific therapeutic regimen should be performed.

By “amplification” is meant the presence of one or more extra gene copies in a chromosome complement.

The word “label” when used herein refers to a compound or composition which is conjugated or fused directly or indirectly to a reagent such as a nucleic acid probe or an antibody and facilitates detection of the reagent to which it is conjugated or fused. The label may itself be detectable (e.g., radioisotope labels or fluorescent labels) or, in the case of an enzymatic label, may catalyze chemical alteration of a substrate compound or composition which is detectable.

The term “antibody” is used in the broadest sense and specifically covers single monoclonal antibodies and antibody compositions with polyepitopic specificity (e.g. polyclonal antibodies) as well as antibody fragments so long as retain their ability to immunospecifically recognize a target polypeptide epitope.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to conventional (polyclonal) antibody preparations which typically include different antibodies directed against different determinants (epitopes), each monoclonal antibody is directed against a single determinant on the antigen. In addition to their specificity, the monoclonal antibodies are advantageous in that they are synthesized by the hybridoma culture, uncontaminated by other immunoglobulins. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al., Nature, 256:495 (1975), or may be made by recombinant DNA methods (see, e.g., U.S. Pat. No. 4,816,567). The “monoclonal antibodies” may also be isolated from phage antibody libraries using the techniques described in Clackson et al., Nature, 352:624-628 (1991) and Marks et al., J. Mol. Biol., 222:581-597 (1991), for example.

As used herein, the term “polynucleotide” means a polymeric form of nucleotides of at least 10 bases or base pairs in length, either ribonucleotides or deoxynucleotides or a modified form of either type of nucleotide, and is meant to include single and double stranded forms of DNA and/or RNA. In the art, this term if often used interchangeably with “oligonucleotide”. A polynucleotide can comprise a nucleotide sequence disclosed herein wherein thymidine (T) can also be uracil (U); this definition pertains to the differences between the chemical structures of DNA and RNA, in particular the observation that one of the four major bases in RNA is uracil (U) instead of thymidine (T).

As used herein, the term “polypeptide” means a polymer of at least about 10 amino acids. Throughout the specification, standard three letter or single letter designations for amino acids are used. In the art, this term is often used interchangeably with “protein”.

The term “primer” or “primers” refers to oligonucleotide sequences that hybridize to a complementary RNA or DNA target polynucleotide and serve as the starting points for the stepwise synthesis of a polynucleotide from mononucleotides by the action of a nucleotidyltransferase, as occurs for example in a polymerase chain reaction.

As used herein, the term “inhibitor” encompasses molecules capable of inhibiting one or more of the biological activities of target molecules such as EGFR polypeptide. Illustrative inhibitors include the targeted small-molecule inhibitors and antibody inhibitors disclosed herein as well as other inhibitors known in the art such as anti-sense polynucleotides and siRNA. Consequently one skilled in the art will appreciate that such inhibitors encompass molecules which inhibit both polynucleotide synthesis and/or function (e.g. antisense polynucleotide molecules) as well those which inhibit polypeptide synthesis and/or function (e.g. molecules which block phosphorylation and hence activity of a target polypeptide such as mTOR). As discussed in detail below, illustrative inhibitors include erlotinib, gefitinib, ZD-1839, OSI-774, PD-153053, PD-168393, IMC-C225, CI-1033, AG1478, 4-(2′fluoroanilino)-and 4-(3′fluoroanilino)-6,7 diethoxyquinazoline, 4-(3′bromoanilino-6,7-dimethoxyquinazoline), AX7593, PP2, pyrrole(2,1-f)(1,2,4) triazine nucleus, 5-substituted-4-hydroxy-8-nitroquinazoline, EKB-569, MSK-039, cetuximab, benzamide, benzamidine, acryloylamino-salicylanilides, and HKI-272.

### Biological Aspects of Embodiments of the Invention

EGFR kinase inhibitors have clinical activity in a relatively small subset of glioblastoma and lung cancer patients. The recent discovery of the strong association between EGFR kinase domain mutations and clinical response in lung cancer patients provides rationale for the use of molecular biomarkers to select patients for EGFR targeted therapy. Because the molecular mechanisms underlying response in glioblastoma have yet to be clarified, enthusiasm for the potential efficacy of EGFR inhibitor therapy for glioblastoma has been limited. Here, we have shown that a subset of recurrent malignant glioma patients treated with gefitinib or erlotinib had >25% tumor shrinkage, significantly prolonged time to progression and overall survival. By demonstrating that EGFRvIII/PTEN co-expression is strongly associated with response to EGFR kinase inhibitors (p=0.00078; relative risk=51.0; 95% CI=(3.89-669)) and by confirming this highly significant association in an independent dataset, we disclose an approach for identifying patients most likely to benefit from gefitinib and erlotinib.

EGFR kinase domain mutants selectively activate anti-apoptotic signals through the PI3K/Akt signaling pathway upon which cancer cells become dependent (see, e.g., Sordella R et al., Science 2004; 305:1163-7). Gefitinib-mediated inhibition of this signal appears to be critical for its efficacy. Like these EGFR kinase domain mutants, EGFRvIII preferentially activates PI3K/Akt pathway signaling (see, e.g., Sordella R et al., Science 2004; 305:1163-7; Choe G et al., Cancer Res 2003; 63:2742-6; Li B et al., Oncogene 2004; 23:4594-602; Huang H S, et al., J Biol Chem 1997; 272:2927-35; and Batra S K et al., Cell Growth Differ 1995; 6:1251-9). Thus our finding of its association with clinical response and its role in sensitizing glioblastoma cells in vitro, is consistent with current thinking on gefitinib sensitivity in lung cancer. However, for EGFR inhibitors to be effective, inhibition of receptor phosphorylation may need to be effectively translated into a diminished PI3K/AKT pathway signal. The best characterized function of PTEN is as a lipid phosphatase that counteracts the growth and survival promoting effects of the PI3K/AKT pathway. PI3K is a lipid kinase that phosphorylates phosphatidylinositols at the 3-position (PtdIns (3, 4, 5) P₃), which subsequently recruit kinases such as AKT (a potent oncogenic survival factor), leading to a cascade of constitutive activation of downstream effectors, including the mammalian Target Of Rapamycin (mTOR) (Vivanco et al.,. Nat Rev Cancer, 2: 489-501, 2002; Hidalgo et al., Oncogene, 19: 6680-6686, 2000; Sawyers et al., Cancer Cell, 4: 343-348, 2003; Fingar et al., Oncogene, 23: 3151-3171, 2004; Luo et al., Cancer Cell, 4: 257-262, 2003). The PTEN tumor suppressor gene encodes a phosphatase that removes the phosphate group from PIP3, thereby regulating the activation state of this pathway. PTEN loss results in constitutive signaling through PIP3, and hence unregulated activation of the Akt pathway.

Thus, PTEN loss, which results in chronic PI3K pathway activation in glioblastoma (see e.g., Choe G et al., Cancer Res 2003; 63:2742-6; and Erinoian R P et al., Clin Cancer Res 2002; 8:1100-6), renders glioblastomas less sensitive to EGFR inhibitors, even if they express EGFRvIII. These results are consistent with recent in vitro studies demonstrating a role for PTEN in determining sensitivity of epithelial cancer cell lines to gefitinib (see, e.g., Bianco R et al., Oncogene 2003; 22:2812-22), and with the recent observation that breast cancer patients with PTEN loss have diminished response to the anti-Her2 monoclonal antibody trastuzurnab (see, e.g., Bianco R et al., Oncogene 2003; 22:2812-22; and Nagata Y et al., Cancer Cell 2004; 6:117-27). Taken together, these results suggest that screening for PTEN protein loss may be warranted in other types of cancer patients with EGFR kinase mutations who do not respond to gefitinib, erlotinib, or related EGFR family kinase inhibitors. As importantly, these studies suggest that downstream inhibition of the PI3K pathway, perhaps at the level of EGFR, may potentially be combined with EGFR inhibitors in PTEN deficient patients in order to promote clinical response.

Monoclonal antibodies and tyrosine kinase inhibitors specifically targeting EGFR and/or EGFRvIII are the most well-studied and hold substantial promise of success. Several compounds of monoclonal antibodies and tyrosine kinase inhibitors targeting EGFR have been studied and clinical trials are now underway to test the safety and efficacy of these targeting strategies in a variety of human cancers. Compounds that target the extracellular ligand-binding region of EGFR include antibodies such as Cetuximab (also known as Erbitux or IMC-C225). Other compounds such as tyrosine kinase inhibitors which target the intracellular domain of EGFR, include ZD-1839 (also known as gefitinib or Iressa), OSI-774 (also known as Erlotinibor or Tarceva), PD-153053, PD-168393 and CI-1033, have been studied in clinical settings alone or in combination with radiation or chemotherapy. In addition, compounds such as h-R3, ABX-EGF, EMD-55900, ICR-62, AG1478, 4-(2′fluoroanilino)-and 4-(3′fluoroanilino)-6,7 diethoxyquinazoline, 4-(3′bromoanilino-6,7-dimethoxyquinazoline), AX7593, PP2, pyrrole(2,1-f)(1,2,4) triazine nucleus, 5-substituted-4-hydroxy-8-nitroquinazoline, EKB-569, MSK-039, cetuximab, benzamide, benzamidine, acryloylamino-salicylanilides, and HKI-272 have proved to be effective in targeting malignant cells alone or in combination with traditional therapies. The effects of ZD 1839 (Iressa) is currently being studied in clinical trails for patients with glioblastoma multiforme. In this context the methods of the invention can be used to examine the PI3K/Akt pathway and then select an appropriate therapeutic agent in cells that do not have a deregulated PI3K/Akt pathway (e.g. an EGFR inhibitor). For discussions of EGFR inhibitors see, e.g. Khalil et al., Expert Rev Anticancer Ther. 2003 June; 3(3):367-80; Chakravarti et al., Int J Radiat Oncol Biol Phys. 2003 Oct. 1; 57(2 Suppl):S329; Wissner et al., Bioorg Med Chem Lett. 2002 Oct. 21; 12(20):2893-7; Ciardiello et al., Expert Opin Investig Drugs, 2002 June; 11(6):755-68; De Bono et al., Trends Mol Med. 2002; 8(4 Suppl):S19-26; and Cohen, Clin Colorectal Cancer. 2003 February; 2(4):246-51; Ellis A G et al., Biochem Pharmacol. 2006 Mar. 4; Hennequin L F et al., Bioorg Med Chem Lett. 2006 Mar. 1; VanBrocklin H F et al., J of Med Chem. 2005 Nov. 17; 48(23):7445-56; Aparna V et al., J Chem Inf Model. 2005 May-June; 45(3):725-38; Shreder K R et al., Org Lett. 2004 Oct. 14; 6(21):3715-8; Li Z et al., Biochem Biophys Res Commun. 2006 Mar. 10; 341(2):363-8; Fink B E et al., Bioorg Med Chem Lett. 2005 Nov. 1; 15(21):4774-9; Hunt J T et al., J Med Chem. 2004 Jul. 29; 47(16):4054-9; Jin Y et al., Bioorg Med Chem. 2005 Oct. 1; 13(19):5613-22; Cavasotto C N et al., Bioorg Med Chem Lett. 2006 Apr. 1; 16(7):1969-74; Li S et al., Cancer Cell. 2005 April; 7(4):301-11; Asano T. et al., Bioorg Med Chem. 2004 Jul. 1; 12(13):3529-42; Deng W et al., Bioorg Med Chem Lett. 2006 Jan. 15; 16(2):469-72; Tsou H R et al., J Med Chem. 2005 Feb. 24; 48(4):1107-31; Lee Y S et al., Arch Pharm. (Weinheim). 2005 October; 338(10):502-5; Kobayashi S et al., Cancer Res. 2005 Aug. 15; 65(16):7096-101; Guo M et al., J Pharmacol Exp Ther. 2005 November; 315(2):526-33; and Mishani E., et al., J Med Chem. 2005 Aug. 11; 48(16):5337-48.

Deregulated or diminished activation of the PI3K/Akt pathway is common in a variety of different cancers (see, e.g. Fresno Vara et al., Cancer Treat Rev. 30(2): 193-204 (2004; Mitsiades et al., Curr. Cancer Drug targets, 4(3): 235-256 (2004); Brader et al., Tumori, 90(1): 2-8 (2004); and Sansal et al., J. Clin. Oncol., 22(14): 2954-2963 (2004). As is known in the art, these cancers are targets of therapeutics including EGFR kinase inhibitors. An illustrative but non limiting list of cancers likely to respond or responsive to EGFR kinase inhibitors, includes glioblastomas and cancers of the prostate (see, e.g., Vivanco et al., Nat Rev Cancer. 2: 489-501, 2002; Feldkamp et al., Journal of Neurooncology 35: 223-248, 1997; Mischel et al., Brain Pathology, January; 13(1):52-61 2003) as well as cancers of the bile duct (see, e.g. Tanno et al., Cancer Res., 64(10): 3486-3490 (2004)), bladder (see, e.g. Riegler-Christ et al., Oncogene, 23(27): 4745-53 (2004), breast (see, e.g. DeGraffenried et al., Ann. Oncol., 15(10): 1510-1516 (2004)), colon (see, e.g. Itoh et al., Cancer, 94(12): 3127-34 (2004)), endometrium (see, e.g. Gagnon et al., Int. J. Oncol., 23(2): 803-10 (2003), leukocytes (see, e.g. Cuni et al., Leukemia, 18(8): 1438-40 (2004); Kubota et al., Leukemia, 18(8): 1391-400 (2004); and Tabelli et al., Br. J. Haematol. 126(4): 574-82 (2004)), liver (see, e.g. Wang et al., Genes Devel. 18(8): 912-25 (2004)), lung (see, e.g. Sithanandam et al., Carcinogenesis 24(10): 1581-92 (2003); and Cappuzzo et al., J. Natl. Cancer Inst. 96(15): 1133-1141 (2004)), melanocytes (see, e.g. Dhawan et al., 62(24): 7335-42 (2002)) ovary (see, e.g. Altomare et al., Oncogene 23(24): 5853-7 (2004)) pancreas (see, e.g. Perugini et al., J. Surg. Res. 90(1): 39-44 (2000), thyroid (see, e.g. Vasko et al., J. Med. Genet. 41(3): 161-70 (2004), esophagus (see, e.g. Sutter et al., Int J Cancer. 2006 Apr. 1; 118(7):1814-22), renal cell (see, e.g., Staehler et al., Curr Drug Targets. 2005 November; 6(7):835-46), hepatocellular carcinoma (see, e.g., Wu T. et al., Cancer Treat Rev. 2006 February; 32(1):28-44), bronchioloalveolar carcinoma (see, e.g., Wislez M et al., Rev Mal Respir. 2005 December; 22(6 Pt 2):8S70-5), nasopharyngeal carcinoma (see, e.g., Lee S C et al., Pharmacogenet Genomics. 2006 January; 16(1):73-4), testicular germ cell tumors (see, e.g., Kollmannsberger C et al., Cancer. 2006 Mar. 15; 106(6):1217-26), gastrointestinal cancer (see, e.g., Macarulla T et al., Onkologie. 2006 March; 29(3):99-105), head and neck (see, e.g., Willmore-Payne C et al., Mod Pathol. 2006 Mar. 17), colorectal cancer (see, e.g., McKenna W G et al., Semin Oncol. 2003 June; 30(3 Suppl 6):56-67), prostate (see, e.g., Festuccia C et al., Endor Relat Cancer. 2005 December; 12(4):983-98), astrocytoma and medulloblastoma (see, e.g., Shelton et al., Expert Opin Ther Targets. 2005 October; 9(5):1009-30), and non-small cell lung carcinoma (see, e.g., Dowell J E et al., Am J Med Sci. 2006 March; 331(3):139-49). Consequently, the assessment of this pathway is critical for stratifying patients for EGFR inhibitor therapy.

As noted above, typical embodiments of the invention examine cellular pathways in the family of tumors termed “gliomas”. Briefly, the brain contains two major cell types: neurons and glia. Glial cells give rise to the family of tumors termed “gliomas”. There are several distinct types of tumors within this glioma grouping. These can range from very benign, slow-growing tumors to rapidly enlarging, highly malignant cancerous types. The most commonly occurring tumors within the glioma family are astocytomas, oligodendroglioma and ependymomas. In addition, some patients may have tumors with a mixed appearance. Astrocytomas are the most common type of glioma. These are tumors that occur within the brain tissue itself. Like all gliomas astrocytomas can be located either superficially or deep within the brain and can affect critical structures. As they arise from the astrocyte cells (which serve as supporting elements of the brain), astrocytomas are generally infiltrative in nature.

As discussed in detail below, the World Health Organization (WHO) grading scheme is used to characterize this group of tumors. Briefly, in the World Health Organization grading system, grade I tumors are the least malignant. These tumors grow slowly and microscopically appear almost normal; surgery alone may be effective. Grade I tumors are often associated with long-term survival. Grade II tumors grow slightly faster than grade I tumors and have a slightly abnormal microscopic appearance. These tumors may invade surrounding normal tissue, and may recur as a grade II or higher tumor. Grade III tumors are malignant. These tumors contain actively reproducing abnormal cells and invade surrounding normal tissue. Grade III tumors frequently recur, often as grade IV tumors. Grade IV tumors are the most malignant and invade wide areas of surrounding normal tissue. These tumors reproduce rapidly, appear very unusual microscopically and are necrotic (have dead cells) in the center. Grade IV tumors cause new blood vessels to form, to help maintain their rapid growth. Glioblastoma multiforme is the most common grade IV tumor. For additional information see, e.g. Tatter S B , Wilson C B, Harsh G R IV. Neuroepithelial tumors of the adult brain. In Youmans J R, ed. Neurological Surgery, Fourth Edition, Vol. 4: Tumors. W.B. Saunders Co., Philadelphia, pp. 2612-2684, 1995; Kleihues P, Burger P C, Scheithauer B W. The new WHO classification of brain tumours. Brain Pathology 3:255-68, 1993; Lopes M B S, VandenBerg S R, Scheithauer B W; The World Health Organization classification of nervous system tumors in experimental neuro-oncology. In A. J. Levine and H. H. Schmidek, eds. Molecular Genetics of Nervous System Tumors Wiley-Liss, New York, pp. 1-36, 1993.

Low-grade astrocytomas (Grades I/IV or II/IV) are termed benign and occur generally in children or young adults. These tumors carry a better prognosis than higher grade astrocytomas. Although the management of these low-grade astrocytomas can be controversial, those tumors which are surgically accessible are usually resected. One of the concerns with low-grade astrocytomas in adults is that they can undergo a malignant transformation and change into a higher-grade, or malignant tumor. The methods of the invention can be used to monitor such transformations. In astrocytomas grade I, normal karyotype is observed most frequently; among the cases with abnormal karyotypes, the most frequent chromosomal abnormalities loss of the X and Y sex-chromosomes; loss of 22q is found in 20-30% of astrocytomas; other abnormalities observed in low grade tumors include gains on chromosome 8q, 10p, and 12p, and losses on chromosomes 1p, 4q, 9p, 11p 16p, 18 and 19.

Anaplastic astrocytomas (Grade III/IV) are more aggressive tumors and, as such, are usually treated in a more radical fashion. In anaplastic astrocytomas, chromosome gains or losses are frequent: trisomy 7 (the most frequent), loss of chromosome 10, loss of chromosome 22, loss of 9p, 13q; other abnormalities, less frequently described are: gains of chromosomes 1q, 11q, 19, 20, and Xq.

Glioblastoma multiforme (Grade IV/IV) is the most malignant form of astrocytomas. Although these tumors can occur at almost any age, the peak incidence is between 50 and 70 years old. Glioblastoma multiforme (GBM) is also called a high-grade glioma and is graded by pathologists as Grade IV/IV astrocytoma. These tumors mostly occur in adults with the peak incidence between 50 and 70 years of age. Generally the time from the onset of symptoms to diagnosis is relatively short, usually just a few weeks. Glioblastomas typically show several chromosomal changes: by frequency order, gain of chromosome 7 (50-80% of glioblastomas), double minute chromosomes, total or partial monosomy for chromosome 10 (70% of tumors) associated with the later step in the progression of glioblastomas partial deletion of 9p is frequent (64% of tumors): 9pter-23; partial loss of 22q in 22q13 is frequently reported loss or deletion of chromosome 13, 13q14-q31 is found in some glioblastomas trisomy 19 was reported in glioblastomas by cytogenetic and comparative genomic hybridization (CGH) analysis; the loss of 19q in 19q13.2-qter was detected by loss of heterozygosity (LOH) studies in glioblastomas deletion of chromosome 4q, complete or partial gains of chromosome 20 has been described; gain or amplification of 12q14-q21 has been reported the loss of chromosome Y might be considered, when it occurs in addition to other clonal abnormalities.

Oligodendrogliomas are benign, slow growing tumors that occur usually in young adults. Often these are located within the frontal lobes which can allow for a safe, complete operative resection. Many oligodendrogliomas contain calcium (little specks of bone) seen best on CT scans.

In summary, EGFRvIII and PTEN are key molecular determinants of glioblastoma sensitivity to EGFR kinase inhibitors. These data disclose that rational application of EGFR kinase inhibitors can increase time to progression and significantly prolong life for a selected subset of glioblastoma patients. Prospective validation of EGFRvIII and PTEN as predictors of clinical response to EGFR kinase inhibitors in independent data sets is warranted. These future studies should also address whether immunohistochemistry or newer molecular based assays are superior for EGFRvIII and PTEN determination in tumor samples.

Further biological aspects of the invention are discussed in the following sections.

### A Subset of Patients Respond to EGFR Inhibitors

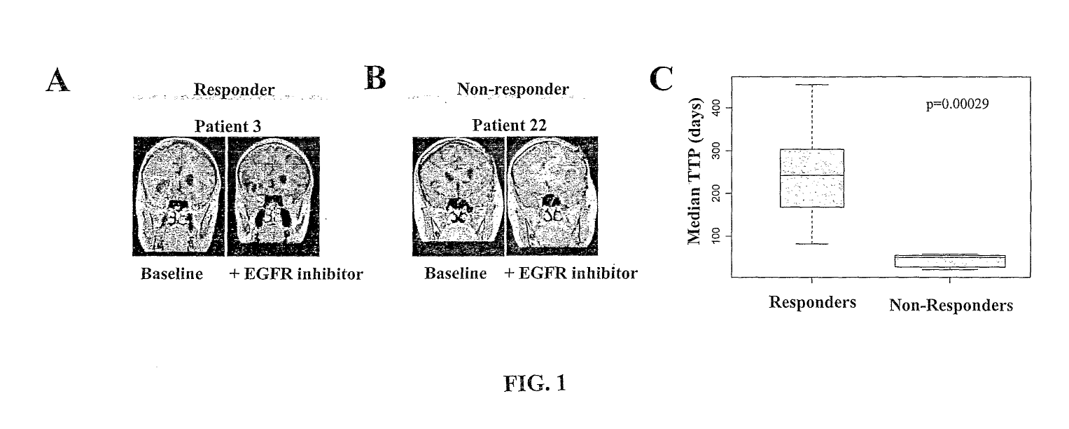

In order to investigate biomarkers that predict response to EGFR kinase inhibitors, we selected the set of patients who demonstrated unequivocal evidence for response or treatment failure (MRI evidence for tumor progression within 8 weeks of initiating therapy). Seven patients had >25% tumor regression as established by bi-directional measurement of the contrast-enhancing tumor on MRI during EGFR inhibitor therapy. Nineteen patients progressed within 8 weeks of treatment with >25% tumor growth on MRI. Clinically, the glioblastoma patients classified as responders by MRI imaging had 4.9 times greater (p=0.00029) median time to progression (243 days, Interquartile range (IQR) (176-300)) relative to non-responders with glioblastoma (50 days, IQR(29-54) (FIG. 1). We reasoned that comparing biopsies from these patients would provide the clearest insight into the molecular correlates of response to EGFR inhibitors. Clinical characteristics of these 26 patients are listed in Table 1. There were no significant correlations between age, gender, extent of surgical resection, Karnofsky performance status or dose of EGFR inhibitor, and treatment response (Table 3).

### EGFR Kinase Domain Mutations Do Not Account for Treatment Response

Given the close association between EGFR kinase domain mutations and response to EGFR kinase inhibitors in lung cancer patients, we sequenced the kinase domain of EGFR. DNA quality was sufficient for sequencing of the entire kinase domain of EGFR (exons 18-24) in 6/7 responders; DNA quality was sufficient for sequencing only exon 21 in patient 6. No mutations were detected in 7/7 responders (Table 2). In addition, no EGFR kinase domain mutations were detected in 8 of the progressive disease patients, for whom DNA was available for sequencing. The sensitivity of this assay to detect EGFR kinase domain mutations was confirmed in a sample of non-EGFR inhibitor treated gliomas and other cancers. Thus, EGFR kinase domain mutations, due to their low frequency, are unlikely to play a major role in determining the sensitivity of glioblastoma patients to EGFR kinase inhibitor therapy. Because mutations in the kinase domain of the EGFR hetetodimerization partner Her2/Neu have been reported in glioblastoma and may affect response to EGFR kinase inhibitors (see, e.g., Stephens P et al., Nature 2004; 431:525-6), we also sequenced the kinase domain of Her2/Neu. No mutations were detected.

EGFR Gene Amplification is not Associated with Response

To exclude the possibility that increased EGFR gene dosage is associated with clinical response to EGFR kinase inhibitors, we assessed EGFR gene amplification by FISH and real-time PCR. EGFR gene amplification was detected in 12/25 (48%) malignant glioma samples (Table 2) and confirmed by real-time PCR, consistent with the previously reported EGFR amplification frequency¹⁸. Seven of 25 (28%) demonstrated polysomy. No association between EGFR gene amplification and response to EGFR inhibitors was detected, in line with previous work (see. e.g., Rich J N et al., J Clin Oncol 2004; 22:133-42).

EGFRvIII and PTEN Protein Coexpression are Significantly Associated with Response

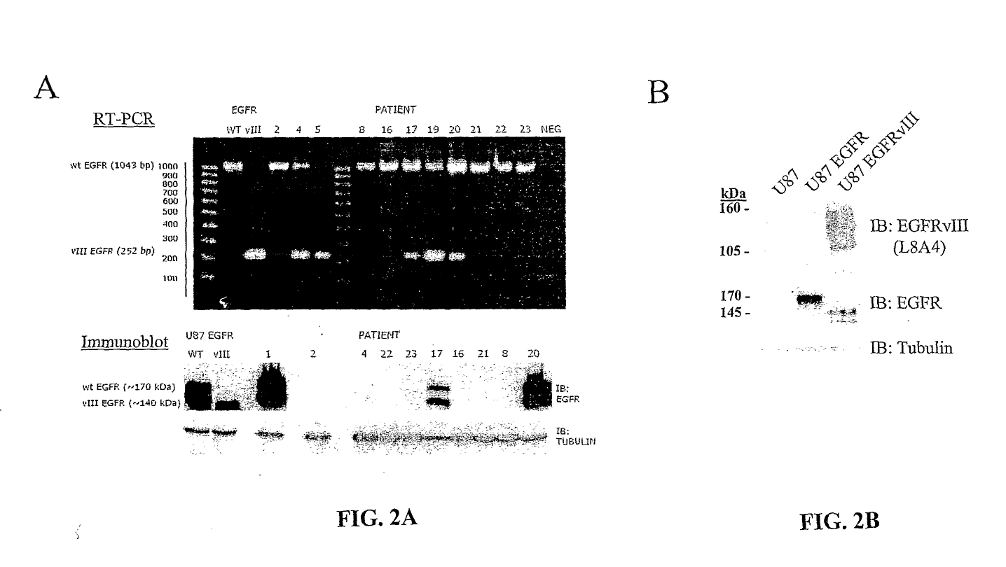

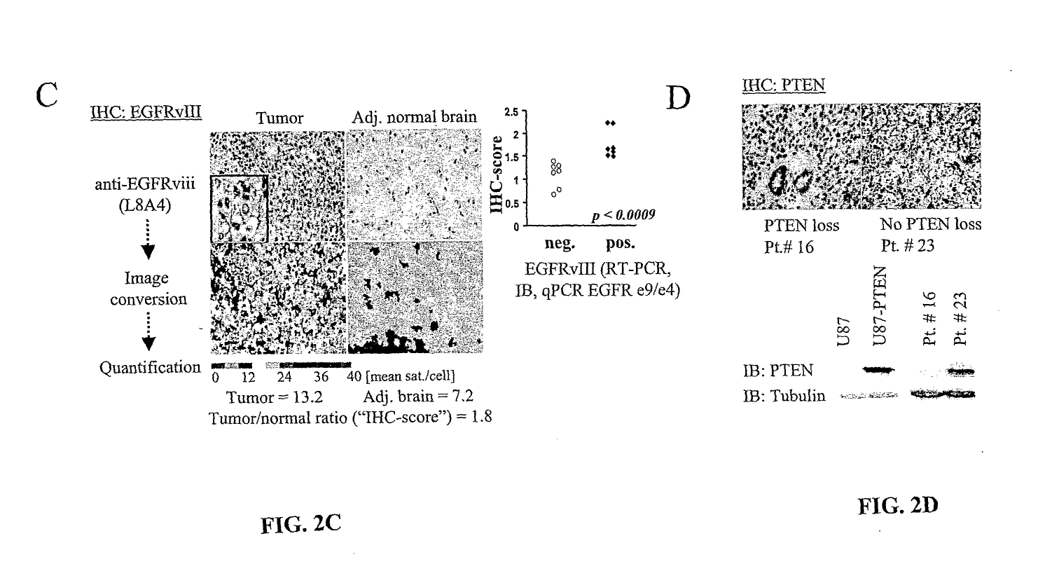

Lacking evidence for activating kinase domain mutations, we focused on the genomic deletion variant EGFRvIII. First, we screened for EGFRvIII by nucleic acid-based assays (RT-PCR and EGFR exon9/4 DNA ratio) and immunoblotting in the 15 patients for whom frozen tissue was available (FIG. 2A, Table 2). We then performed EGFRvIII immunohistochemistry using an antibody that specifically recognizes EGFRvIII, but not wild-type EGFR (FIG. 2B) (see, e.g., Wikstrand C J et al., Cancer Res 1997; 57:4130-40). EGFRvIII immunohistochemistry was scored semiquantitatively by two pathologists blinded to the molecular analyses, and by quantitative image analysis (FIG. 2C, supplementary methods). We found complete agreement between the nucleic acid-based assays and immunohistochemistry (κ=1.0; p=0.00031), as well as between immunoblotting and immunohistochemistry (κ=1.0; p=0.00091). These results are consistent with recent findings by another group (see, e.g., Aldape K D et al., J Neuropathol Exp Neurol 2004; 63:700-7), and justified the use of immunohistochemistry for determination of EGFRvIII status in the remaining 11 patients for whom no frozen tissue was available.

EGFRvIII was detected in 12/26 cases (46%) (Table 2), similar to the previously reported frequency (see, e.g., Rich J N et al., J Clin Oncol 2004; 22:133-42). EGFRvIII was found only in patients whose tumors had EGFR gene amplification or whole chromosome 7 gain; EGFRvIII was not detected in any patients with normal EGFR copy number. Six of twelve patients (50%) whose tumors expressed EGFRvIII responded to EGFR inhibitors; by contrast, only 1/14 (7%) patients lacking EGFRvIII expression responded to EGFR inhibitors (p<0.027 by a two sided Fisher's exact test). These data suggested that EGFRvIII expression may sensitize malignant glioma patient to EGFR kinase inhibitors. However, the lack of clinical response in 50% of patients with EGFRvIII expressing tumors raised the possibility that other factors also influence clinical response.

Recent pre-clinical models suggest that PTEN protein expression may be required for response to EGFR family kinase inhibitors (see, e.g., Bianco R et al., Oncogene 2003; 22:2812-22; and Nagata Y et al., Cancer Cell 2004; 6:117-27). To determine whether PTEN loss renders glioblastoma resistant to EGFR kinase inhibitors, we analyzed PTEN protein expression by immunohistochemistry and immunoblotting (FIG. 2D). Both molecular assays for PTEN determination correlated highly for tumors for which frozen tissue was available (κ=0.8; p=0.0047). Zero of thirteen (0%) patients whose tumors showed PTEN protein loss responded to EGFR inhibitors; in contrast, 7/13 (54%) of PTEN-positive tumors responded (p<0.0053 by a two sided Fisher's exact test) (Table 2). To exclude the possibility that the immunohistochemical assay was detecting a mutant protein, we sequenced PEEN. No mutations were detected in the responders. Thus, wild type PTEN protein expression appears to be critical for response to EGFR inhibitors. More importantly, patients whose tumors co-expressed EGFRvIII and PTEN were 51 times more likely to respond to EGFR inhibitor therapy (95% CI=(3.89-669); p=0.00078) relative to those patients whose tumors lacked expression of both proteins (Table 3). An EGFRvIII/PTEN co-expression molecular diagnostic for clinical response had a sensitivity of 75% and specificity of 94% in this data set.

Having determined that EGFRvIII/PTEN co-expression is significantly associated with response, we turned our attention to a subset of patients who did not have either tumor regression or substantial tumor growth while on EGFR inhibitor therapy. These ten patients were excluded from the original analysis because they did not fit the extremes of clear response or treatment failure (i.e. they did not have a <25% or >25% change in tumor size on MRI). None of these patients (0%) had tumors that co-expressed EGFRvIII and PTEN protein. Thus, an EGFRvIII/PTEN co-expression based biomarker of response would have predicted that these patients will not respond well to therapy. Accordingly, these patients had a poor outcome with median time to progression of 112 days; IQR=(88-138). This result further demonstrates the potential robustness of the EGFRvIII/PTEN co-expression based predictor of response to EGFR kinase inhibitors.

### Independent Validation of EGFRvIII/PTEN as Biomarkers of Response

To confirm these findings in a set of patients treated at a different institution, we analyzed EGFRvIII/PTEN expression in biopsies from 33 malignant glioma patients treated at UCSF with erlotinib (Supplementary Table 1). UCSF requited a minimum of 50% tumor shrinkage in order for a patient to be coded as a responder, as compared to the 25% shrinkage required in the UCLA study. Nonetheless, patients whose tumors co-expressed EGFRvIII and PTEN were 16 times more likely to respond to erlotinib (95% CI=(2.0-128); p=0.01) (Table 3). An EGFRvIII/PTEN co-expression molecular diagnostic for clinical response had a sensitivity of 67% and specificity of 89%. Thus, the association between EGFRvIII/PTEN co-expression and response to EGFR inhibitors is robust and replicable.

### EGFRvIII and PTEN Co-Expression Sensitizes Glioblastoma Cells to EGFR Inhibitors

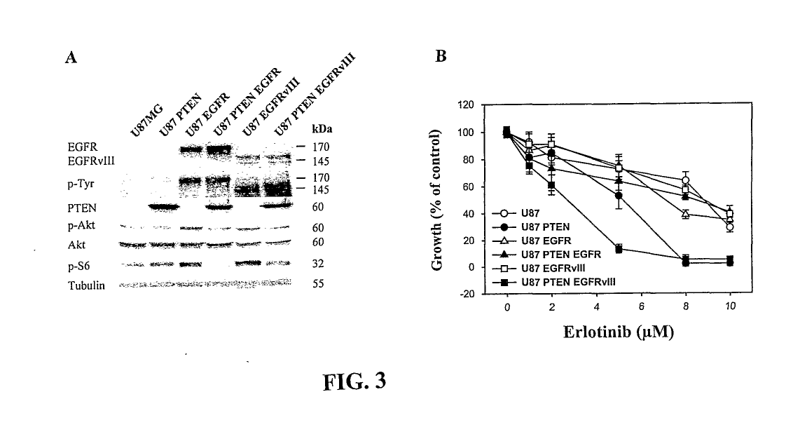

To determine if the correlation between EGFRvIII/PTEN co-expression and clinical response had a mechanistic basis, we expressed PTEN, EGFR and EGFRvIII in relevant combinations in U87MG glioblastoma cells. U87MG cells are PTEN deficient (see, e.g. Steck P A et al., Nat Genet 1997; 15:356-62; Li J et al., Science 1997; 275:1943-7; Furnari F B et al., Cancer Res 1998; 58:5002-8; and Furnari F B et al., Proc Natl Acad Sci USA 1997; 94:12479-84), express low levels of wild type EGFR, and lack EGFRvIII protein (see, e.g., Han Y et al., Cancer Res 1996; 56:3859-61; and Mishima K et al., Cancer Res 2001; 61:5349-54). Following retroviral transduction we selected the following stable cell lines: U87-PTEN, U87-EGFR, U87-PTEN-EGFR, U87-EGFRvIII and U87-PTEN-EGFRvIII. Multiple clones were analyzed and clones stably expressing the relevant proteins at similar levels between the cell lines were used for subsequent analyses (FIG. 3). Consistent with our findings in the glioblastoma patient samples, EGFRvIII sensitized glioblastoma cells to erlotinib, but only when PTEN was co-expressed.

### Typical Methods of the Invention

As described herein, the status (e.g. coexpression, expression of only one or expression or neither) of EGFRvIII and PTEN polypeptides and/or polynucleotides in cells of a patient suffering from or suspected of suffering from a cancer such as a glioma may be evaluated in by a variety of methods well known in the art. The evaluation of the status of EGFRvIII and PTEN provides information useful in diagnostic and prognostic protocols, for example to determine whether the cancer cell is likely to respond, or is responsive to an epidermal growth factor receptor (EGFR) inhibitor. In these methods the status of the EGFRvIII and PTEN genes are examined by any of a number of art accepted protocols such as a genomic Southerns to evaluate gross perturbations of genomic DNA, Northern and PCR analysis to evaluate the presence and levels of EGFRvIII and PTEN mRNAs or immunological methods to examine the presence and levels of EGFRvIII and PTEN proteins. Such protocols are typically used to examine the coexpression of EGFRvIII and PTEN. Alternatively, such protocols can be used to examine presence or absence of mutations within EGFRvIII and PTEN mRNA or proteins.

Another embodiment of the invention is a method of determining the sensitivity of a mammalian tumor cell to an epidermal growth factor receptor (EGFR) inhibitor comprising examining the tumor cell for the presence of EGFRvIII and PTEN proteins. In specific illustrative embodiments of these methods, the coexpression of EGFRvIII and PTEN is examined by a protocol selected from the group consisting of Southern hybridization, Northern hybridization, immunohistochemistry, immunoblotting, polymerase chain reaction and polynucleotide sequencing. In a more specific embodiment, the mammalian tumor cell does not express EGFR having a deletion mutation in the kinase domain, does not express HER2 (SEQ ID NO:3) having a deletion mutation in the kinase domain, and/or does not exhibit amplification of the gene that encodes EGFR (SEQ ID NO:4). In a preferred embodiment of this method, the coexpression of EGFRvIII and PTEN is examined by evaluating the presence or levels of EGFRvIII and PTEN mRNA transcripts within the cell. In another preferred embodiment, the cell analyzed in this method is from a biopsied tissue sample. In a specific embodiment of this method, the test cell is a human cell. In a more specific embodiment of this method, the test cell is suspected of being a tumor cell. In a highly preferred embodiment, the test cell suspected of being a tumor is a glioma such as glioblastoma.

As discussed in detail herein, the status of EGFRvIII and PTEN gene products in patient samples can be analyzed by a variety protocols that are well known in the art including, inarriunohistochemical analysis, the variety of Northern blotting techniques including in situ hybridization, RT-PCR analysis (for example on laser capture micro-dissected samples), Western blot analysis, immunohistochemistry and tissue array analysis. More particularly, the invention provides assays for the evaluation of EGFRvIII and PTEN polynucleotides in a biological sample, such brain, and other tissues, cell preparations, and the like. EGFRvIII and PTEN polynucleotides which can be evaluated include, for example, a EGFRvIII and PTEN gene or fragment thereof, and EGFRvIII and PTEN mRNAs. A number of methods for amplifying and/or detecting the presence of EGFRvIII and PTEN polynucleotides are well known in the art and can be employed in the practice of this aspect of the invention.

In one embodiment, a method for detecting EGFRvIII and PTEN mRNAs in a cell comprises producing cDNA from the sample by reverse transcription using at least one primer; amplifying the cDNA so produced using EGFRvIII and PTEN polynucleotides as sense and antisense primers to amplify EGFRvIII and PTEN cDNAs therein; and detecting the presence of the amplified EGFRvIII and PTEN cDNAs. Optionally, the sequence of the amplified EGFRvIII and PTEN cDNAs can be determined.

In another embodiment, a method of detecting a EGFRvIII and PTEN genes in a cell comprises first isolating genomic DNA from the sample; amplifying the isolated genomic DNA using EGFRvIII and PTEN polynucleotides as sense and antisense primers; and detecting the presence (or absence) of the amplified EGFRvIII and PTEN genes. Any number of appropriate sense and antisense probe combinations can be designed from the nucleotide sequences of EGFRvIII and PTEN and used for this purpose.

The invention also provides assays for detecting the presence of EGFRvIII and PTEN proteins in cells or tissues such as brain and other tissues, and the like. Methods for detecting a EGFRvIII and PTEN proteins are also well known and include, for example, immunoprecipitation, immunohistochemical analysis, Western blot analysis, molecular binding assays, ELISA, ELIFA and the like. For example, a method of detecting the presence or levels of EGFRvIII and PTEN proteins in a biological sample comprises first contacting the sample with EGFRvIII and PTEN antibody, a EGFRvIII and PTEN-reactive fragment thereof, or a recombinant protein containing an antigen binding region of a EGFRvIII and/or PTEN antibody; and then detecting the binding of EGFRvIII and PTEN protein in the sample.

Significantly, the disclosed methods for examining these biomarkers are useful with a wide variety of tissue samples including formalin fixed, paraffin embedded biopsy samples. As described herein, these markers can be examined using antibodies. In these methods, a mammalian cell such as a cell derived from a formalin fixed, paraffin embedded biopsy sample can be examined for evidence of EGFRvIII and PTEN coexpression by examining a tissue sample containing this cell for the presence of these molecules. Certain embodiments of the invention identify and/or assess a therapeutic agent that may be used to treat the glioma such as an EGFR inhibitor (e.g. erlotinib, gefitinib, ZD-1839, OSI-774, PD-153053, PD-168393, IMC-C225, CI-1033, AG1478, 4-(2′fluoroanilino)-and 4-(3′fluoroanilino)-6,7 diethoxyquinazoline, 4-(3′bromoanilino-6,7-dimethoxyquinazoline), AX7593, PP2, pyrrole(2,1-f)(1,2,4) triazine nucleus, 5-substituted-4-hydroxy-8-nittoquinazoline, EKB-569, MSK-039, cetuximab, benzamide, benzamidine, acryloylamino-salicylanilides, and HKI-272).

Typically the assays of the invention include immunohistochemical techniques. Immunohistochemical techniques as used herein encompasses the use of reagents detecting cell specific markers, such reagents include, for example antibodies. Antibodies, including monoclonal antibodies, polyclonal antibodies and fragments thereof; are often used to identify proteins or polypeptides of interest in a sample. A number of techniques are utilized to label objects of interest according to immunohistochemical techniques. Such techniques are discussed in Current Protocols in Molecular Biology, Unit 14 et seq., eds. Ausubel, et al., John Wiley & Sons, 1995, the disclosure of which is incorporated herein by reference. Typical protocols include staining a paraffin embedded tissue section prepared according to a conventional procedure (see, e.g. U.S. Pat. No. 6,631,203).

### Typical Protocols Useful to the Practice of the Invention

### 1. Antibodies

The antibodies useful in the invention may comprise polyclonal antibodies, for example affinity purified polyclonal antibodies. Methods of preparing polyclonal antibodies are known to the skilled artisan. Polyclonal antibodies can be raised in a mammal, for example, by one or more injections of an immunizing agent and, if desired, an adjuvant. Typically, the immunizing agent and/or adjuvant will be injected in the mammal by multiple subcutaneous or intraperitoneal injections. The immunizing agent may include the appropriate polypeptide epitopes (e.g. a S6 polypeptide (SEQ ID NO: 5) having a phosphorylated serine, threonine or tyrosine residue, a ERK polypeptide (SEQ ID NO: 7) having a phosphorylated serine, threonine or tyrosine residue, a AKT polypeptide (SEQ ID NO: 6) having a phosphorylated serine, threonine or tyrosine residue, or a PTEN polypeptide) or a fusion protein thereof.

In addition, it may be useful to conjugate the immunizing agent to a protein known to be immunogenic in the mammal being immunized. Examples of such immunogenic proteins include but are not limited to keyhole limpet hemocyanin, serum albumin, bovine thyroglobulin, and soybean trypsin inhibitor. Examples of adjuvants which may be employed include Freund's complete adjuvant and MPL-TDM adjuvant (monophosphoryl Lipid A, synthetic trehalose dicorynomycolate). The immunization protocol may be selected by one skilled in the art without undue experimentation.

The antibodies may, alternatively, be monoclonal antibodies. Monoclonal antibodies may be prepared using hybridoma methods, such as those described by Kohler and Milstein, Nature, 256:495 (1975). In a hybridoma method, a mouse, hamster, or other appropriate host animal, is typically immunized with an immunizing agent to elicit lymphocytes that produce or are capable of producing antibodies that will specifically bind to the immunizing agent. Alternatively, the lymphocytes may be immunized in vitro.

The immunizing agent will typically include a phosphorylated S6, ERK or AKT polypeptide or a fusion protein thereof. Generally, either peripheral blood lymphocytes (“PBLs”) are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell (Goding, Monoclonal Antibodies: Principles and Practice, Academic Press, (1986) pp. 59-103). Immortalized cell lines are usually transformed mammalian cells, particularly myeloma cells of rodent, bovine and human origin. Usually, rat or mouse myeloma cell lines are employed. The hybridoma cells may be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells.

Preferred immortalized cell lines are those that fuse efficiently, support stable high level expression of antibody by the selected antibody-producing cells, and are sensitive to a medium such as HAT medium. More preferred immortalized cell lines are murine myeloma lines, which can be obtained, for instance, from the Salk Institute Cell Distribution Center, San Diego, Calif. and the American Type Culture Collection, Rockville, Md. Human myeloma and mouse-human heteromyeloma cell lines also have been described for the production of human monoclonal antibodies (Kozbor, J. Immunol., 133:3001 (1984); Brodeur et al., Monoclonal Antibody Production Techniques and Applications, Marcel Dekker, Inc., New York, (1987) pp. 51-63).

The culture medium in which the hybridoma cells are cultured can then be assayed for the presence of monoclonal antibodies directed against phosphorylated S6, ERK or AKT polypeptides or PTEN and EGFR polypeptides. Preferably, the binding specificity of monoclonal antibodies produced by the hybridoma cells is determined by immunoprecipitation or by an in vitro binding assay, such as radioimmunoassay (RIA) or enzyme-linked immunoabsorbent assay (ELISA). Such techniques and assays are known in the art. The binding affinity of the monoclonal antibody can, for example, be determined by the Scatchard analysis of Munson and Pollard, Anal. Biochem., 107:220 (1980).

After the desired hybridoma cells are identified, the clones may be subcloned by limiting dilution procedures and grown by standard methods (Goding, supra). Suitable culture media for this purpose include, for example, Dulbecco's Modified Eagle's Medium and RPMI-1640 medium. Alternatively, the hybridoma cells may be grown in vivo as ascites in a mammal. The monoclonal antibodies secreted by the subclones may be isolated or purified from the culture medium or ascites fluid by conventional immunoglobulin purification procedures such as, for example, protein A-Sepharose, hydroxylapatite chromatography, gel electrophoresis, dialysis, or affinity chromatography.

The monoclonal antibodies may also be made by recombinant DNA methods, such as those described in U.S. Pat. No. 4,816,567. DNA encoding the monoclonal antibodies of the invention can be readily isolated and sequenced using conventional procedures (e.g., by using oligonucleotide probes that are capable of binding specifically to genes encoding the heavy and light chains of murine antibodies). The hybridoma cells of the invention serve as a preferred source of such DNA. Once isolated, the DNA may be placed into expression vectors, which are then transfected into host cells such as simian COS cells, Chinese hamster ovary (CHO) cells, or myeloma cells that do not otherwise produce immunoglobulin protein, to obtain the synthesis of monoclonal antibodies in the recombinant host cells. The DNA also may be modified, for example, by substituting the coding sequence for human heavy and light chain constant domains in place of the homologous murine sequences (U.S. Pat. No. 4,816,567; Morrison et al., supra) or by covalently joining to the immunoglobulin coding sequence all or part of the coding sequence for a non-immunoglobulin polypeptide. Such a non-immunoglobulin polypeptide can be substituted for the constant domains of an antibody of the invention, or can be substituted for the variable domains of one antigen-combining site of an antibody of the invention to create a chimeric bivalent antibody.

The antibodies may be monovalent antibodies. Methods for preparing monovalent antibodies are well known in the art. For example, one method involves recombinant expression of immunoglobulin light chain and modified heavy chain. The heavy chain is truncated generally at any point in the Fc region so as to prevent heavy chain crosslinking. Alternatively, the relevant cysteine residues are substituted with another amino acid residue or are deleted so as to prevent crosslinking.

In vitro methods are also suitable for preparing monovalent antibodies. Digestion of antibodies to produce fragments thereof, particularly, Fab fragments, can be accomplished using routine techniques known in the art.

Reactivity of antibodies with the cognate protein can be established by a number of well known means, including Western blot, immunoprecipitation, ELISA, and FACS analyses. A antibody or fragment thereof can be labeled with a detectable marker or conjugated to a second molecule. Suitable detectable markers include, but are not limited to, a radioisotope, a fluorescent compound, a bioluminescent compound, chemiluminescent compound, a metal chelator or an enzyme.

### 2. Assays

The invention provides assays for examining cellular pathways associated with disregulated cell growth. Certain embodiments of the invention include the steps of detecting the presence of EGFRvIII and PTEN or phosphorylated S6, AKT or ERK polypeptides or PTEN and EGFR polypeptides in a tissue. Methods for detecting these polypeptides are well known and include, for example, immunoprecipitation, immunohistochemical analysis, Western blot analysis, molecular binding assays, ELISA, ELIFA and the like.

In preferred embodiments of the invention, the expression of PTEN and EGFRvIII proteins in a sample is examined using Immunohistochemical staining protocols. Immunohistochemical staining of tissue sections has been shown to be a reliable method of assessing alteration of proteins in a heterogeneous tissue. Immunohistochemistry (IHC) techniques utilize an antibody to probe and visualize cellular antigens in situ, generally by chromogenic or fluorescent methods. This technique excels because it avoids the unwanted effects of disaggregation and allows for evaluation of individual cells in the context of morphology. In addition, the target protein is not altered by the freezing process.

Preferred protocols that examine the expression of PTEN and EGFRvIII proteins in a sample typically involve the preparation of a tissue sample followed by immunohistochemistry. Illustrative protocols are provided below.

### Sample Preparation

For sample preparation, any tissue sample from a subject may be used. Examples of tissue samples that may be used include, but are not limited to, brain, colon, breast, prostate, ovary, lung, endometrium, stomach, salivary gland or pancreas. The tissue sample can be obtained by a variety of procedures including, but not limited to surgical excision, aspiration or biopsy. The tissue may be fresh or frozen. In one embodiment, the tissue sample is fixed and embedded in paraffin or the like.

The tissue sample may be fixed (i.e. preserved) by conventional methodology (See e.g., “Manual of Histological Staining Method of the Armed Forces Institute of Pathology,” 3^{rd} edition (1960) Lee G. Luna, H T (ASCP) Editor, The Blakston Division McGraw-Hill Book Company, New York; The Armed Forces Institute of Pathology Advanced Laboratory Methods in Histology and Pathology (1994) Ulreka V. Mikel, Editor, Armed Forces Institute of Pathology, American Registry of Pathology, Washington, D.C.). One of skill in the art will appreciate that the choice of a fixative is determined by the purpose for which the tissue is to be histologically stained or otherwise analyzed. One of skill in the art will also appreciate that the length of fixation depends upon the size of the tissue sample and the fixative used. By way of example, neutral buffered formalin, Bouin's or paraformaldehyde, may be used to fix a tissue sample.

Generally, the tissue sample is first fixed and is then dehydrated through an ascending series of alcohols, infiltrated and embedded with paraffin or other sectioning media so that the tissue sample may be sectioned. Alternatively, one may section the tissue and fix the sections obtained. By way of example, the tissue sample may be embedded and processed in paraffin by conventional methodology (See e.g., “Manual of Histological Staining Method of the Armed Forces Institute of Pathology”, supra). Examples of paraffin that may be used include, but are not limited to, Paraplast, Broloid, and Tissuemay. Once the tissue sample is embedded, the sample may be sectioned by a microtome or the like (See e.g., “Manual of Histological Staining Method of the Armed Forces Institute of Pathology”, supra). By way of example for this procedure, sections may range from about three microns to about five microns in thickness. Once sectioned, the sections may be attached to slides by several standard methods. Examples of slide adhesives include, but are not limited to, silane, gelatin, poly-L-lysine and the like. By way of example, the paraffin embedded sections may be attached to positively charged slides and/or slides coated with poly-L-lysine.

If paraffin has been used as the embedding material, the tissue sections are generally deparaffinized and rehydrated to water. The tissue sections may be deparaffinized by several conventional standard methodologies. For example, xylenes and a gradually descending series of alcohols may be used (See e.g., “Manual of Histological Staining Method of the Armed Forces Institute of Pathology”, supra). Alternatively, commercially available deparaffinizing non-organic agents such as Hemo-De7 (CMS, Houston, Tex.) may be used.

### Immunohistochemistry (IHC)

Subsequent to tissue preparation, a tissue section may be subjected to IHC. IHC may be performed in combination with additional techniques such as morphological staining and/or fluorescence in-situ hybridization as disclosed in the examples.

Two general methods of IHC are available; direct and indirect assays. According to the first assay, binding of antibody to the target antigen is determined directly. This direct assay uses a labeled reagent, such as a fluorescent tag or an enzyme-labeled primary antibody, which can be visualized without further antibody interaction. In a typical indirect assay, unconjugated primary antibody binds to the antigen and then a labeled secondary antibody binds to the primary antibody. Where the secondary antibody is conjugated to an enzymatic label, a chromogenic or fluorogenic substrate is added to provide visualization of the antigen. Signal amplification occurs because several secondary antibodies may react with different epitopes on the primary antibody.

The primary and/or secondary antibody used for immunohistochemistry typically will be labeled with a detectable moiety. Numerous labels are available which can be generally grouped into the following categories:

(a) Radioisotopes, such as ³⁵S, ¹⁴C, ¹²⁵I, ³H, and ¹³¹I. The antibody can be labeled with the radioisotope using the techniques described in Current Protocols in Immunology, Volumes 1 and 2, Coligen et al., Ed. Wiley-Interscience, New York, N.Y., Pubs. (1991) for example and radioactivity can be measured using scintillation counting.

(b) Colloidal gold particles.

(c) Fluorescent labels including, but are not limited to, rare earth chelates (europium chelates), Texas Red, rhodamine, fluorescein, dansyl, Lissamine, umbelliferone, phycocrytherin, phycocyanin, or commercially available fluorophores such SPECTRUM ORANGE7 and SPECTRUM GREEN7 and/or derivatives of any one or more of the above. The fluorescent labels can be conjugated to the antibody using the techniques disclosed in Current Protocols in Immunology, supra, for example. Fluorescence can be quantified using a fluorimeter.

Various enzyme-substrate labels are available and U.S. Pat. No. 4,275,149 provides a review of some of these. The enzyme generally catalyzes a chemical alteration of the chromogenic substrate that can be measured using various techniques. For example, the enzyme may catalyze a color change in a substrate, which can be measured spectrophotometrically. Alternatively, the enzyme may alter the fluorescence or chemiluminescence of the substrate. Techniques for quantifying a change in fluorescence are described above. The chemiluminescent substrate becomes electronically excited by a chemical reaction and may then emit light which can be measured (using a chemiluminometer, for example) or donates energy to a fluorescent acceptor. Examples of enzymatic labels include luciferases (e.g., firefly luciferase and bacterial luciferase; U.S. Pat. No. 4,737,456), luciferin, 2,3-dihydrophthalazinediones, malate dehydrogenase, urease, peroxidase such as horseradish peroxidase (HRPO), alkaline phosphatase, β-galactosidase, glucoamylase, lysozyme, saccharide oxidases (e.g., glucose oxidase, galactose oxidase, and glucose-6-phosphate dehydrogenase), heterocyclic oxidases (such as uricase and xanthine oxidase), lactoperoxidase, microperoxidase, and the like. Techniques for conjugating enzymes to antibodies are described in O'Sullivan et al., Methods for the Preparation of Enzyme-Antibody Conjugates for use in Enzyme Immunoassay, in Methods in Enzym. (ed. J. Langone & H. Van Vunakis), Academic press, New York, 73:147-166 (1981).

Examples of enzyme-substrate combinations include, for example:

Horseradish peroxidase (HRPO) with hydrogen peroxidase as a substrate, wherein the hydrogen peroxidase oxidizes a dye precursor (e.g., orthophenylene diamine (OPD) or 3,3′,5,5′-tetramethyl benzidine hydrochloride (TMB));
(ii) alkaline phosphatase (AP) with para-Nitrophenyl phosphate as chromogenic substrate; and
β-D-galactosidase (β-D-Gal) with a chromogenic substrate (e.g., p-nitrophenyl-β-D-galactosidase) or fluorogenic substrate (e.g., 4-methylumbelliferyl-β-D-galactosidase).

Numerous other enzyme-substrate combinations are available to those skilled in the art. For a general review of these, see U.S. Pat. Nos. 4,275,149 and 4,318,980. Sometimes, the label is indirectly conjugated with the antibody. The skilled artisan will be aware of various techniques for achieving this. For example, the antibody can be conjugated with biotin and any of the four broad categories of labels mentioned above can be conjugated with avidin, or vice versa. Biotin binds selectively to avidin and thus, the label can be conjugated with the antibody in this indirect manner. Alternatively, to achieve indirect conjugation of the label with the antibody, the antibody is conjugated with a small hapten and one of the different types of labels mentioned above is conjugated with an anti-hapten antibody. Thus, indirect conjugation of the label with the antibody can be achieved.

Aside from the sample preparation procedures discussed above, further treatment of the tissue section prior to, during or following IHC may be desired. For example, epitope retrieval methods, such as heating the tissue sample in citrate buffer may be carried out (see, e.g., Leong et al. Appl. Immunohistochem. 4(3):201 (1996)).

Following an optional blocking step, the tissue section is exposed to primary antibody for a sufficient period of time and under suitable conditions such that the primary antibody binds to the target protein antigen in the tissue sample. Appropriate conditions for achieving this can be determined by routine experimentation. The extent of binding of antibody to the sample is determined by using any one of the detectable labels discussed above. Preferably, the label is an enzymatic label (e.g. HRPO) which catalyzes a chemical alteration of the chromogenic substrate such as 3,3′-diaminobenzidine chromogen. Preferably the enzymatic label is conjugated to antibody which binds specifically to the primary antibody (e.g. the primary antibody is rabbit polyclonal antibody and secondary antibody is goat anti-rabbit antibody).

Specimens thus prepared may be mounted and coverslipped. Slide evaluation is then determined, e.g. using a microscope.

Where the antigen is an antigen such as PTEN and EGFRvIII, staining intensity criteria may be evaluated as described in the Examples.

### Further Embodiments of the Invention

Further embodiments of the invention follow. These embodiments are related to those disclosed in U.S. patent application Ser. No. 10/701,490 filed Nov. 5, 2003. As is known in the art, another method for identifying whether a mammalian tumor cell is likely to respond, or is responsive to a therapy such as epidermal growth factor receptor (EGFR) inhibitor therapy, is to measure the phosphorylated state of the proteins in the PI3K/AKT pathway. Artisians know that the PI3K/AKT pathway is frequently disregulated in cancers, leading to uncontrolled cell growth. Proteins in this pathway are constitutively phosphorylated and therefore continuously activated in cancer. Measuring the phosphorylated state of these proteins can be a method of determining therapeutic efficacy. Specifically, the cell can be examined for phosphorylated S6 ribosomal polypeptide (SEQ ID NO:5); phosphorylated AKT polypeptide (SEQ ID NO:6); or phosphorylated ERK polypeptide (SEQ ID NO:7), wherein increased phosphorylation of AKT, ERK, and/or S6 ribosomal polypeptide compared to a control identifies the cell as not likely to respond/non-responsive to therapy, such as EGFR inhibitor therapy.

As noted above, the disclosure provided herein identifies a series of biomarkers that are associated with deregulated activation of the PI3K/Akt pathway, a pathway whose deregulated activation is common in cancers such as gliomas. The disclosure provided herein further describes a method of identifying these biomarkers as is known in the art. Since artisans know that this growth related pathway is common pathway that is distegulated in a wide variety of human cancers, artisans understand that the methods and materials described herein can be universally applied to examine this pathway in all cancers in which the deregulated activation of the PI3K/Akt pathway is observed.

Significantly, these biomarkers are useful with a wide variety of tissue samples including formalin-fixed and paraffin-embedded biopsy samples. As is known in the art, these markers can be examined using a panel of antibodies such as phospho-specific antibodies. In these methods, a mammalian cell such as a cell derived from a formalin fixed, paraffin embedded glioblastoma multiforme biopsy sample can be examined for evidence of Akt pathway activation by examining a tissue sample containing this cell for the presence of the various target molecules including phosphorylated polypeptides.

The methods and reagents can be used to determine the activation state of biomarker polypeptides such as Akt and its downstream effectors such as mTOR, ERK, Forkhead and S6-kinase on routinely processed patient biopsy samples (e.g. glioblastoma samples) and this information can be used to determine whether patients will respond to EGFR inhibitors.

The activation of the PI3′K/Akt pathway can be detected with phospho-specific antibodies in routinely processed patient biopsies. In one illustrative embodiment of the invention, the disclosed methods and materials can be used to examine glioblastomas. In another illustrative embodiment, activation of these signal transduction pathways can have prognostic importance. For example, it is known that primary GBM patients whose tumors are activated downstream of Akt, or at the level of ERK, have significantly shorter time to tumor progression and significantly diminished overall survival. After determination of the molecular subtypes of GBMs artisans can stratify patients for targeted molecular therapy.

GBMs are among the most heterogeneous tumors, as has been previously shown (see, e.g., Cheng et al., J Neuropathol Exp Neurol. 58: 120-8., 1999; Jung et al., J Neuropathol Exp Neurol. 58: 993-9., 1999). This poses a problem for assessment of molecular alterations in GBMs, as well as for stratification of patients for targeted inhibitor therapy. Using the disclosure provided herein and methods typically employed in the art one can directly determine the extent of intra-tumor molecular heterogeneity for PTEN, EGFR and EGFRvIII and assess the impact of this on pathway activation, prognosis and response to therapy.

Typically, the methods of the invention are used in evaluating the whether a tumor such as a glioma is likely to respond (i.e. is likely to exhibit growth inhibition) when contacted with an EGFR inhibitor. In such embodiments, the tumor is examined prior to its exposure to the inhibitor. Alternatively, the methods evaluate whether a tumor such as a glioma or prostate cancer is responsive (i.e. exhibits growth inhibition) to an EGFR inhibitor. In such embodiments, the activity of a biomarker polypeptide that is associated with the activation of a pathway (e.g. a phosphorylated S6 ribosomal polypeptide (SEQ ID NO: 5)) can be examined after the tumor is exposed to the inhibitor to determine if the biomarkers in the pathway responded to inhibitor exposure. The art teaches that this growth related pathway is a common pathway that is disregulated in a wide variety of human cancers. Consequently, artisans understand that the methods and materials disclosed herein can be universally applied to examine this pathway in all cancers in which the deregulated activation of the PI3K/Akt pathway is observed. In this context, while the use of the disclosed methods and materials in the examination of gliomas represents the preferred embodiment of the invention, artisans understand that this is an illustrative embodiment and that these methods and materials can be applied to a wide variety of human cancers.

In typical methods, the expression of the biomarker polypeptides is examined using an antibody such as an antibody that binds an epitope comprising a phosphorylated serine residue at position 235 in SEQ ID NO: 5, an antibody that binds an epitope comprising a phosphorylated serine residue at position 473 in SEQ ID NO: 6, or an antibody that binds an epitope comprising a phosphorylated threonine residue at position 202 and tyrosine 204 in SEQ ID NO: 7. Optionally, the sample is a paraffin embedded biopsy sample.

As noted above, certain embodiments of the invention include the examination of the expression of a polypeptide or phosphorylation of a polypeptide. As is known in the art, the examination of such polypeptide expression and polypeptide phosphorylation status in a cell or tissue sample is typically evaluated as compared to a control, i.e. a control cell and/or tissue sample that has a defined or predetermined level of polypeptide expression or phosphorylation. In an example of polypeptide phosphorylation, a control can be a normal tissue (e.g. non-cancerous glial cells) where it is observed that a polypeptide is typically not phosphorylated.

Another embodiment of the invention is a method for determining the responsiveness of a mammalian cancer cell to a growth inhibitory agent selected from the group consisting of a EGFR polypeptide (SEQ ID NO: 4) inhibitor, the method comprising examining the glioblastoma cell for the presence of a S6 polypeptide (SEQ ID NO: 5) having a phosphorylated serine, threonine or tyrosine residue; a AKT polypeptide (SEQ ID NO: 6) having a phosphorylated serine, threonine or tyrosine residue; or a ERK polypeptide (SEQ ID NO: 7) having a phosphorylated serine, threonine or tyrosine residue, wherein the presence of a phosphorylated S6, AKT or ERK polypeptide, determines the responsiveness of the mammalian cancer cell to the growth inhibitory agent. Optionally in such methods, the mammalian cancer cell has been contacted with the growth inhibitory agent. Alternatively, the mammalian cancer cell has not been contacted with the growth inhibitory agent.

As noted above, embodiments of the invention typically utilize antibodies that specifically bind phosphorylated polypeptides, i.e. polypeptides having a phosphorylated serine, threonine or tyrosine residue. In this context the disclosure provides antibodies that bind to specific epitopes comprising a phosphorylated residue. By utilizing antibodies that bind to an epitope that comprises a phosphorylated residue (i.e. phospho-specific antibodies) but which do not bind to the unphosphorylated form of the same polypeptide, these phospho-specific antibodies can be used to examine the activation status of a pathway, where the activation is associated with phosphorylation of one or more specified residues. In certain embodiments of the invention, the phosphorylation status and/or expression levels of multiple members of a signaling pathway (e.g. S6 and AKT) are examined as a confirmatory assessment of the signaling cascade associated with the pathway.

Certain embodiments of the invention are used with formalin fixed, paraffin embedded biopsy samples. In particular, the disclosure provided herein demonstrates that antibodies such as phospho-specific antibodies can be used with antigen samples processed in this manner. Significantly, the disclosure provided herein further demonstrates that the methods using these samples provide an accurate demonstration of the physiological status of the pathways in these samples. Consequently, the disclosure provided herein demonstrates how the methods of the invention are well suited for use with commonly available clinical samples.

In one illustrative embodiment of the invention, the presence of a S6 polypeptide (SEQ ID NO: 5) having a phosphorylated serine, threonine or tyrosine residue is examined using an antibody that binds an epitope comprising a phosphorylated serine residue at position 235 in SEQ ID NO: 5. In another illustrative embodiment of the invention, the presence of a AKT polypeptide (SEQ ID NO: 6) having a phosphorylated serine, threonine or tyrosine residue is examined using an antibody that binds an epitope comprising a phosphorylated serine residue at position 473 in SEQ ID NO: 6.

The methods of the present invention typically utilize antibodies directed to polypeptides in the PI3K/Akt pathway or antibodies directed to EGFRvIII and PTEN. Illustrative antibody compositions useful in the present invention are anti-phosphoprotein antibodies characterized as containing antibody molecules that specifically immunoreacts with a phosphorylated form of a polypeptide associated with the PI3K/Akt pathway. The polypeptide may be for example, S6, AKT or ERK. By “specifically immunoreacts”, it is meant that the antibody binds to the phosphorylated form of polypeptide (i.e. is phospho-specific) and does not bind to the unphosphorylated form of the same polypeptide. Consequently, the phosphorylation associated with pathway activation can be examined with such antibodies. Therefore, the antibodies of the invention can distinguish between the phosphorylated and unphosphorylated forms of a polypeptides associated with the PI3K/Akt pathway. Consequently, the phosphorylation associated with pathway activation can be examined with such antibodies. Typically the assays of the invention include immunohistochemical techniques using the antibodies disclosed herein. For example, a sample can be examined for the presence of a biochemical pathway associated phosphorylated polypeptide such as phosphorylated ERK by using an antibody that binds an epitope comprising a phosphorylated threonine residue at position 202 and tyrosine 204 in SEQ ID NO: 7.

### Articles of Manufacture of the Invention

Embodiments of the invention also include articles of manufacture and/or kits designed to facilitate the methods of the invention. Typically such kits include instructions for using the elements therein according to the methods of the present invention. Such kits can comprise a carrier means being compartmentalized to receive in close confinement one or more container means such as vials, tubes, and the like, each of the container means comprising one of the separate elements to be used in the method. For example, one of the containers can comprise one or more EGFRvIII and PTEN probes herein (e.g. EGFRvIII and PTEN antibodies and/or polynucleotide probes and primers) that is or can be detectably labeled with a marker. For kits utilizes immunological methods (e.g. immunohistochemistry and Western blotting) to detect the target proteins, the kit can also have containers containing buffers for these methods and/or containers comprising antibodies labeled with a reporter-means, such as a chromophore or radioactive molecule.

In a typical embodiment of the invention, an article of manufacture containing materials useful for the examination of the disorders described above is provided. The article of manufacture comprises a container and a label. Suitable containers include, for example, bottles, vials, syringes, and test tubes. The containers may be formed from a variety of materials such as glass or plastic. The container can hold a composition (e.g. a polynucleotide probe and/or antibody composition) which is effective for examining mammalian cells (e.g. glioma cells). The label on, or associated with, the container indicates that the composition is used for examining cellular polypeptides. The article of manufacture may further comprise a second container comprising a buffer, such as phosphate-buffered saline, Ringer's solution and dextrose solution. It may further include other materials desirable from a commercial and user standpoint, including other buffers, diluents, filters, needles, syringes, and package inserts with instructions for use.

Yet another embodiment of the invention is a kit for characterizing a mammalian a cancer such as glioblastoma (GBM) tumor or cell, the kit comprising: an antibody that binds PTEN polypeptide (SEQ ID NO: 1) and an antibody that binds EGFRvIII polypeptide (SEQ ID NO: 2). Optionally the kit further comprises at least one reagent for using these antibodies and instructions for use. A related embodiment of the invention is a kit for characterizing a mammalian cancer such as a glioblastoma (GBM) tumor or cell, the kit comprising: an polynucleotide that hybridizes to PTEN (SEQ ID NO: 8) and a polynucleotide that hybridizes EGFRvIII (SEQ ID NO: 9). Optionally the kit further comprises at least one reagent for using these polynucleotides and instructions for use.

Throughout this application, various publications are referenced. The disclosures of these publications are hereby incorporated by reference herein in their entireties.

### Examples

The Examples below provide illustrative methods and materials that can be used in the practice of the invention.

UCLA patient samples: Forty-nine patients with recurrent malignant glioma have been treated at UCLA since 2001 with gefitinib (n=37) or erlotinib (n=12) as part of three UCLA IRB approved multi-institutional clinical trials. Two trials were performed through Cancer Therapy Evaluation Program (NIH); one was industry sponsored. Diagnoses were established by two board-certified neuropathologists and confirmed independently by a pathologist blinded to the molecular analyses. Tumor specimens were obtained during diagnostic or surgical procedures according to a UCLA IRB approved protocol. All patients had measurable disease by magnetic resonance imaging (MRI), and had been off previous treatments (for at least 4 weeks) at the start of EGFR inhibitor monotherapy. MRI and clinical assessment were performed at baseline, two-month intervals, and at the time of progression by a neuroradiologist and neuro-oncologist blinded to the molecular analyses. Thirty-seven patients, 26 of whom had clear-cut evidence of either response or progression, had sufficient tissue for molecular analysis. One patient was excluded because response occurred coincident with increase in decadron. See supplement for statistical methods.

UCSF patient samples. We obtained paraffin-embedded slides from biopsies of 33 malignant glioma patients treated at UCSF with erlotinib. No frozen tissue was available from these patients. EGFRvIII and PTEN immunohistochemistry were scored semiquantitatively by two UCLA pathologists blinded to the clinical information. A prediction of treatment response based on the immunohistochemical results was sent to UCSF prior to unblinding the clinical response data.

Sequencing of tumor genomic DNA. Exons and flanking intronic sequences for EGFR (kinase domain), HER2/neu (kinase domain) and PTEN (all exons) were amplified using specific primers in a 384-well format nested PCR setup, as performed by Agencourt Bioscience Corporation (Beverly, Mass.). See supplementary methods for details. High quality sequence variations found in one or both directions were scored as candidate mutations. Exons harboring candidate mutations were re-amplified from the original DNA sample and re-sequenced as above. Primer sequences have been published elsewhere (see, e.g., Paez J G et al., Science 2004; 304:1497-500).

Fluorescence in situ hybridization: Dual probe fluorescence in situ hybridization (FISH) was performed on paraffin-embedded sections with locus specific probes for EGFR and centromere of chromosome 7 (CEP7) (Vysis, Downers Grove, Ill.). Standard FISH protocols for pretreatment, hybridization and analyses were followed per manufacturer's suggestions (see, e.g., Smith J S et al., J Natl Cancer Inst 2001; 93:1246-56).

RT-PCR: High quality total RNA was extracted from 13 fresh frozen tumor samples (4 responders, 9 non-responders). Complementary DNA (cDNA) was synthesized and amplified using primers designed to specifically amplify EGFR (1043 bp product) and EGFRvIII (252 bp product). See supplementary methods for details.

Real-time PCR: Genomic DNA from fifteen samples (7 responders, 8 nonresponders) was extracted and real-time PCR was performed using the iCycler thermocycler (Bio-Rad Laboratories). All measurements were collected in triplicate and confirmed by independent experiments. See supplementary methods for primer sequences used and complete details.

Immunohistochemistry and immunoblotting: PTEN and EGFRvIII immunohistochemistry were performed on paraffin-embedded tissue sections, and scored by two independent pathologists blinded to the molecular analyses. Scores of 0 and 1 were considered PTEN loss, as previously reported (see, e.g., Choe G et al., Cancer Res 2003; 63:2742-6). In cases where heterogeneity of PTEN staining was present, tumors with diminished or absent staining in at least 25% of the section were considered deficient. Tumors demonstrating at least focal moderate to strong EGFRvIII immunostaining were considered positive, as previously reported²⁵. Quantitative image analysis to confirm the pathologist-based scoring was also performed using the Soft Imaging System software (see supplementary methods for complete details).

Functional analysis of effect of EGFR, EGFRvIII and PTEN coexpression: Human EGFR and EGFRvIII cDNAs were retrovirally introduced into U87MG and U87MG-PTEN over-expressing cells and selected for antibiotic resistance. Multiple stable clones were analyzed for the expression of PTEN, EGFR or EGFRvIII by immunoblot. To determine relative sensitivity to erlotinib, 1000 cells/well were seeded into 96-well plates in 8 replicates. Twenty-four hours later, erlotinib was added at final concentrations ranging from 0-10 μM. Plates were incubated for 10-14 days, fixed and stained with 0.25% crystal violet in methanol and quantified. The background reading of the wells containing medium alone was subtracted from experimental wells. Experiments were repeated 3 times in 8 replicates for each condition, and similar results were obtained. Multiple clones for each cell line confirmed these results.

### Sequencing of Tumor Genomic DNA

Each PCR reaction contained 5 ng of DNA, 1× HotStar Buffer, 0.8 mM dNTPs, 1 mM MgCl₂, 0.2U HotStar Enzyme (Qiagen, Valencia, Calif.), and 0.2 μM forward and reverse primers in a 10 μL, reaction volume. PCR cycling parameters were: one cycle of 95° C. for 15 min, 35 cycles of 95° C. for 20 s, 60° C. for 30 s and 72° C. for 1 min, followed by one cycle of 72° C. for 3 min. The resulting PCR products were purified by solid phase reversible immobilization chemistry followed by bi-directional dye-terminator fluorescent sequencing with universal M13 primers. Sequencing fragments were detected via capillary electrophoresis using ABI Prism 3700 DNA Analyzer (Applied Biosystems, Foster City, Calif.). PCR and sequencing were performed by Agencourt Bioscience Corporation (Beverly, Mass.). Forward (F) and reverse (R) chromatograms were analyzed in batch by Mutation Surveyor 2.03 (SoftGenetics, State College, Pa.), followed by manual review. High quality sequence variations found in one or both directions were scored as candidate mutations. Exons harboring candidate mutations were re-amplified from the original DNA sample and re-sequenced as above.

RT-PCR: Total RNA was extracted from 50-100 mg of each frozen tumor specimen using TRIzol reagent (Invitrogen) in a tissue grinder. Total RNA was then treated with amplification grade DNase I for 15 minutes at 37° C. For each patient, 1 microgram of total RNA was reverse transcribed using Superscript II (Invitrogen) with oligo(dT) priming according to the manufacturer's protocols. 2 microliters of 1st strand cDNA ( 1/10th of the reverse transcription reaction volume) was then used as template in a 50 microliter PCR reaction containing 2 mM MgSO₄, 0.2 M of each primer, 0.2 mM dNTPs, 1× High Fidelity PCR Buffer, and 1 unit of Platinum Taq High Fidelity (Invitrogen). Forward and reverse primer sequences to specifically amplify EGFR and EGFRvIII were 5′ CTT CGG GGA GCA GCG ATG CGA C 3′ (SEQ ID NO: 18) (spanning the 5′ untranslated region and the beginning of exon 1) and 5′ ACC AAT ACC TAT TCC GTT ACA C 3′ (SEQ ID NO: 10) (within exon 9), respectively. These primers generate a 1043 by PCR product for the wild type EGFR transcript compared to a 252 by PCR product for the EGFRvIII transcript. PCR cycling conditions began with an initial denaturation step at 95° C. for 2 minutes, followed by 42 cycles of 95° C. denaturation for 30 seconds, 56.5° C. annealing for 30 seconds, and 68° C. extension for 1:20. PCR reactions were analyzed by running 5 μL of product on a 1.5% agarose gel and staining with ethidium bromide. Cloned wild type EGFR and EGFRvIII cDNAs were used as templates in parallel positive control reactions, alongside reverse transcription and PCR negative control reactions. GAPDH was also amplified for each patient sample to assess relative RNA template quality and amount. Primers for GAPDH were 5′ GTG AAG GTC GGA GTC AAC GG 3′ (SEQ ID NO: 19) and 5′ TGA TGA CAA GCT TCC CGT TCT C 3′ (SEQ ID NO: 11) (generating a 198 bp product) and the extension time during cycling was reduced to 30 seconds.

Real-time PCR: Real-time PCR was performed using the iCycler thermocycler (Bio-Rad Laboratories). Amplification conditions were: 95° C. for 3 min., 40 cycles of 95° C./30 sec and 72° C./1 min., and 75 cycles of 63° C.+0.5° C. per cycle for 5 sec for melt curve analyses. Each amplification reaction contained 10 ng of tumor DNA, 101AM of each primer, Titanium Taq polymerase, 1× Titanium Taq buffer (Clontech), 125 μm dNTP, SYBR™ Green I (Molecular Probes), and Fluorescein (Bio-Rad Laboratories). Normal human genomic DNA (Promega) was used as control DNA template. To control for variations in input DNA between tumor samples, GAPDH amplifications were performed in parallel with EGFR exon 4 and EGFR exon 9 amplifications and used for subsequent normalization. All measurements were collected in triplicates and confirmed by independent experiments. Primers for realtime-PCR included: EGFR exon 4: forward, AAAGAGTGCTCACCGCAGTT (SEQ ID NO: 12), reverse, CACTGGATGCTCTCCACGTT (SEQ ID NO: 13); EGFR exon 9: forward, CTTCAAAAACTGCACCTCCA (SEQ ID NO: 14), reverse, CAAGCAACTGAACCTGTGACT (SEQ ID NO: 15); GAPDH: forward, CAGCAAGAGCACAAGAGGAA (SEQ ID NO: 16), reverse, CAACTGTGAGGAGGGGAGAT (SEQ ID NO: 17).

Immunoblotting: Snap-frozen tissues or cell culture cells were lysed and homogenized in RIPA lysis buffer containing fresh protease inhibitors by standard procedures. Protein concentrations were quantified with the BCA Protein Assay kit (Pierce Chemical Co), and 30 fig of proteins were separated in 8% SDS-PAGE gel, transferred to nitrocellular membranes, and hybridized with antibodies to the indicated antigens by standard procedures. Signals were detected by chemoluminescence using ECL detection reagents (Amersham Pharmacia Biotech). Primary antibodies to the following antigens were used: EGFR/EGFRvIII cocktail (#AHR5062, Biosource Corp., Camarillo, Calif., USA), phospho-Tyr (#9411, Cell Signaling), PTEN (#ABM-2052, Cascade), Akt (#9272, Cell Signaling), phospho-Akt (Ser473/587F11, #4051, Cell Signaling), S6 (#2212, Cell Signaling), phospho-S6 (Ser235/236, #2211, Cell Signaling), and β-tubulin (T4026, Sigma).

Immunohistochemistry: Sections were stained with monoclonal antibodies to PTEN (clone 6H2.1, Cascade Bioscience, Winchester Mass.) and EGFRvIII (clone L8A4, a generous gift from Dr. Darrell Bigner). L8A4 has been shown to react with EGFRvIII, but not full-length EGFR (see, e.g., Wikstrand C J et al., Cancer Res 1997; 57:4130-40). Antigen retrieval was performed using 0.01 M citrate buffer, pH 6.0 for 30 minutes in an oven. Peroxidase activity was quenched with 3% hydrogen peroxide in water. Primary antibodies (PTEN at 1:400, EGFRvIII at 1:150) were diluted in phosphate buffered saline with 2% bovine serum albumin and 2% normal horse serum and applied for 16 hours at 4° C., followed by biotinylated secondary antibodies (Vector) at 1:200 dilution for 30 minutes, and avidin-biotin complex (Elite ABC, Vector) for 30 minutes. Negative control slides received blocking serum (phosphate buffered saline with 2% normal horse serum and 2% bovine serum albumin). Vector NovaRed was used as the enzyme substrate to visualize specific antibody localization. Slides were counterstained with Harris hematoxylin.

Pathologist based scoring of immunohistochemistry: PTEN staining was scored according to a previously established scale of 0-2, which has been shown to be highly consistent (see, e.g., Choe G et al., Cancer Res 2003; 63:2742-6; and Choe G et al., Cancer Res 2003; 63:2742-6). Tumor cells are graded as 2 if their staining intensity is equal to that of the vascular endothelium, 1 if it is diminished relative to the endothelium, and 0 if it is undetectable in the tumor cells and present in the vascular endothelium. Tumors with PTEN scoring of 0 or 1 are considered PTEN deficient. EGFRvIII staining was scored as positive for tumors demonstrating at least focal moderate to strong immunoreactivity, as previously reported (see. e.g., Choe G et al., Cancer Res 2003; 63:2742-6). Tumors were scored for PTEN and EGFRvIII by two independent neuropathologists, blinded to the molecular analyses.

Image analysis-based scoring of immunohistochemistry: Representative images from PTEN and EGFRvIII immunostained sections were photographed using a Colorview II camera mounted on an Olympus BX61 microscope. Multiple images were captured (at least 3 per slide) from representative regions of the tumor (and adjacent normal brain if present). Borders between individual cells were approximated using a filter function. The amount of reaction product per cell was determined by measuring mean saturation per cell in the red-brown hue range. 1000-1500 cells per case (on average) were measured for EGFRvIII and PTEN. As an internal control, for PTEN analysis, mean saturation was measured in vascular endothelium; for EGFRvIII analysis, mean saturation was measured in adjacent normal brain tissue. For samples in which no adjacent normal brain was present on the slide, a normal reference standard was established by analyzing 9700 cells from 15 normal brain sections. Ratios of mean PTEN staining per tumor cell/mean PTEN staining per endothelial cell; and mean EGFRvIII staining per tumor cell/mean EGFRvIII staining per normal brain were determined. False color images representing the distribution of such cells were generated. For PTEN staining, a tumor/vessel ratio <0.6 was considered to be PTEN loss. The agreement between traditional semi-quantitative pathologic assessment and image analysis was very high (kappa=0.92; p=0.000006) For EGFRvIII, the correlation between semi-quantitative pathologic assessment and image analysis was also very high (kappa=0.91; p=0.000007).

Statistical Methods: To test the dependence of 2 categorical variables (corresponding to the rows and columns of a contingency table), we used Fisher's exact test. We used a logistic regression model to estimate the odds ratio (relative risk) and its confidence interval between 2 binary variables. To test whether ordinal variables differed across 2 groups, we used the Wilcoxon or the Kruskal-Wallis test, both of which are non-parametric group comparison tests. To measure agreement between categorical measurements, we used Cohen's kappa statistic, which takes on values smaller than or equal to 1 (=perfect agreement). The asymptotic standard error of the kappa statistic can be used to arrive at an asymptotic p-value, which measures the significance of agreement. We used the Spearman correlation coefficient and the corresponding p-value to determine the correlation between quantitative or ordinal variables. The Kaplan-Meier (KM) method was used to estimate survival distributions. The Cox proportional hazards model was used to estimate hazard rates, their confidence intervals, and corresponding Cox regression p-values. All p-values were two sided and p<0.05 was considered significant. All statistical analyses were carried out with the freely available software (e.g. as found at http://www.r-project.org/).

The present invention is not to be limited in scope by the embodiments disclosed herein, which are intended as single illustrations of individual aspects of the invention, and any that are functionally equivalent are within the scope of the invention. Various modifications to the models and methods of the invention, in addition to those described herein, will become apparent to those skilled in the art from the foregoing description and teachings, and are similarly intended to fall within the scope of the invention. Such modifications or other embodiments can be practiced without departing from the true scope and spirit of the invention.

### Tables

TABLE 1
Patient Characteristics

| | | | | | | | MRI response | | Overall |
|---|---|---|---|---|---|---|---|---|---|
| Patient | | | | EGFR | Dose | | % change in | TTP | Survival |
| No. | Sex | Age | Diagnosis | Inhibitor | (mg) | EIAED | TV | (days) | (days) |
| RESPONDERS | | | | | | | | | |
| 1 | M | 47 | GBM | Erlotinib | 300-500 | Yes | −82 | 290 | >372 |
| 2 | M | 60 | GBM | Erlotinib | 150-200 | No | −57 | 303 | >380 |
| 3 | F | 19 | GBM | Gefitinib | 1500-1000 | Yes | −44 | 195 | >459 |
| 4 | M | 27 | AO** | Gefitinib | 500 | No | −57 | 999 | >999 |
| 5 | F | 41 | GBM | Gefitinib | 500-250 | No | −87 | 456 | 742 |
| 6 | F | 60 | GBM | Gefitinib | 500-750 | No | −50 | 169 | 235 |
| 7* | M | 65 | GBM | Erlotinib | 150-200 | No | −35 | NA* | 82* |
| NON-RESPONDERS | | | | | | | | | |
| 8 | M | 50 | GBM | Gefitinib | 150 | No | 35 | 27 | 182 |
| 9 | F | 39 | GBM | Erlotinib | 150-200 | No | 100 | 54 | 103 |
| 10 | M | 64 | GBM | Erlotinib | 300-450 | Yes | 106 | 54 | 111 |
| 11 | F | 56 | GBM | Gefitinib | 500-750 | No | 121 | 47 | 156 |
| 12 | M | 38 | GBM | Gefitinib | 500 | No | 697 | 27 | 182 |
| 13 | M | 46 | GBM | Erlotinib | 300-350 | Yes | 38 | 23 | 23 |
| 14 | M | 40 | GBM | Erlotinib | 150 | No | 453 | 54 | 318 |
| 15 | M | 60 | GBM | Gefitinib | 500-1000 | No | 628 | 57 | 145 |
| 16 | M | 58 | GBM | Erlotinib | 150 | No | 105 | 52 | 174 |
| 17 | M | 57 | GBM | Gefitinib | 500 | No | 319 | 40 | 167 |
| 18 | F | 42 | GBM | Gefitinib | 500 | No | 450 | 53 | 318 |
| 19 | M | 55 | GBM | Gefitinib | 150 | No | 150 | 34 | 173 |
| 20 | F | 52 | GBM | Gefitinib | 150-200 | No | 200 | 54 | 184 |
| 21 | M | 39 | GBM | Gefitinib | 150 | Yes | 87 | 20 | 201 |
| 22 | F | 31 | AO** | Gefitinib | 500 | No | 418 | 55 | 105 |
| 23 | F | 32 | GBM | Gefitinib | 500 | No | 182 | 42 | 190 |
| 24 | F | 41 | GBM | Gefitinib | 500 | No | 332 | 26 | 198 |
| 25 | F | 58 | GBM | Erlotinib | 150-200 | No | 34 | 54 | 186 |
| 26 | M | 26 | GBM | Gefitinib | 500 | No | 350 | 54 | 65 |

*This patient died of unrelated cardiac arrythmia during response. Minimal residual tumor was found at autopsy.
**hese two patients were excluded from time to progression analysis.

SUPPLEMENTARY TABLE 1
PTEN and EGFRvIII status in validation set

| Patient | | | | | Dose | | | PTEN | EGFRvIII |
|---|---|---|---|---|---|---|---|---|---|
| No. | Sex | Age | Diagnosis | Drug | (mg) | Temozolide* | EAIED | IHC | IHC |
| RESPONDERS | | | | | | | | | |
| 1 | M | 50 | GBM | Erlotinib | 400 | No | Yes | No loss | Present |
| 2 | M | 74 | GBM | Erlotinib | 150 | No | No | No loss | Present |
| 3 | M | 40 | GBM | Erlotinib | 350 | No | Yes | Loss | Present |
| 4 | M | 45 | Oligo | Erlotinib | 250 | Yes | Yes | No loss | Present |
| 5 | F | 56 | GBM | Erlotinib | 300 | No | Yes | Loss | Present |
| 6 | M | 47 | AA | Erlotinib | 400 | No | yes | No loss | Present |
| NON-RESPONDERS | | | | | | | | | |
| 7 | F | 53 | GBM | Erlotinib | 100 | Yes | No | Loss | Present |
| 8 | F | 53 | GBM | Erlotinib | 100 | No | Yes | Loss | Absent |
| 9 | F | 44 | GBM | Erlotinib | 250 | No | Yes | Loss | Present |
| 10 | M | 64 | GBM | Erlotinib | 400 | No | Yes | Loss | Absent |
| 11 | F | 58 | GBM | Erlotinib | 150 | No | Yes | Loss | Absent |
| 12 | F | 47 | GBM | Erlotinib | 250 | No | No | Loss | Present |
| 13 | M | 65 | GBM | Erlotinib | 300 | No | Yes | Loss | Absent |
| 14 | F | 60 | GBM | Erlotinib | 150 | No | No | Loss | Present |
| 15 | F | 63 | GBM | Erlotinib | 100 | Yes | Yes | Loss | Present |
| 16 | F | 72 | GBM | Erlotinib | 200 | Yes | Yes | Loss | Present |
| 17 | F | 58 | GBM | Erlotinib | 100 | No | Yes | Loss | Absent |
| 18 | F | 28 | AO | Erlotinib | 250 | No | Yes | No loss | Absent |
| 19 | F | 68 | GBM | Erlotinib | 100 | No | No | Loss | Present |
| 20 | F | 60 | GBM | Erlotinib | 200 | No | Yes | Loss | Absent |
| 21 | M | 38 | AA | Erlotinib | 100 | No | No | Loss | Absent |
| 22 | F | 42 | GBM | Erlotinib | 250 | Yes | Yes | Loss | Absent |
| 23 | M | 34 | GBM | Erlotinib | 450 | No | Yes | Loss | Absent |
| 24 | M | 41 | Oligo | Erlotinib | 400 | No | Yes | No loss | Absent |
| 25 | M | 58 | GBM | Erlotinib | 350 | No | Yes | Loss | Absent |
| 26 | M | 50 | GBM | Erlotinib | 150 | No | No | Loss | Present |
| 27 | M | 53 | AA | Erlotinib | 200 | No | No | No loss | Absent |
| 28 | M | 60 | GBM | Erlotinib | 500 | No | Yes | Loss | Absent |
| 29 | M | 58 | AA | Erlotinib | 250 | No | No | No loss | Present |
| 30 | F | 50 | GBM | Erlotinib | 200 | Yes | No | No loss | Present |
| 31 | F | 35 | GBM | Erlotinib | 250 | No | No | Loss | Absent |
| 32 | F | 44 | AA | Erlotinib | 250 | No | No | Loss | Present |
| 33 | M | 66 | GBM | Erlotinib | 500 | No | Yes | Loss | Present |

*Concurrent Temozolomide therapy (150 mb/m2 daily for 5 days followed by 3 days off in repeating 28 day cycles). Temozolomide increased in 200 mg/m2 if not hematological toxicity was encountered.

TABLE 2
EGFR and PTEN status in tumor tissue

| | EGFR | | | | | |
|---|---|---|---|---|---|---|
| Patient | K.D. | | EGFRvIII | | | PTEN |
| No. | mutations | FISH | IHC | RT-PCR | Immunoblot | IHC |
| RESPONDERS | | | | | | |
| 1 | Neg | AMP | Pos | ND | Pos | No loss |
| 2 | Neg | NON AMP | Neg | Neg | Neg | No loss |
| 3 | Neg | POLY | Pos | NA | NA | No loss |
| 4 | Neg | POLY | Pos | Pos | Pos | No loss |
| 5 | Neg | POLY | Pos | Pos | NA | No loss |
| 6 | Neg | AMP | Pos* | NA | NA | No loss |
| 7 | Neg | AMP | Pos | Pos | Pos | No loss |
| NON-RESOPNDERS | | | | | | |
| 8 | Neg | NON AMP | Neg | Neg | Neg | No loss |
| 9 | NA | POLY | Neg | NA | NA | Loss |
| 10 | NA | AMP | Pos | NA | NA | No loss |
| 11 | NA | AMP | Neg | NA | NA | Loss |
| 12 | NA | AMP | Neg | NA | NA | Loss |
| 13 | NA | AMP | Neg | NA | NA | Loss |
| 14 | Neg | POLY | Neg | Neg | Neg | No loss |
| 15 | NA | AMP | Neg | NA | NA | Loss |
| 16 | Neg | NON AMP | Neg | Neg | Neg | Loss |
| 17 | Neg | AMP | NT | Pos | Pos | No loss |
| 18 | NA | POLY | Pos | NA | NA | Loss |
| 19 | NA | AMP | Pos | Pos | NA | Loss |
| 20 | Neg | AMP | Pos | Pos | Pos | Loss |
| 21 | Neg | POLY | Neg | Neg | Neg | Loss |
| 22 | Neg | NON AMP | Neg | Neg | Neg | Loss |
| 23 | Neg | NON AMP | Neg | Neg | Neg | No loss |
| 24 | NA | NON AMP | Neg | NA | NA | No loss |
| 25 | NA | AMP | Pos | NA | NA | Loss |
| 26 | NA | NT | Neg | NA | NA | Loss |

Abbreviations: K.D. = kinase domain; FISH = fluorescence in situ hybridization; AMP = amplified; NON-AMP = non-amplified/diploid; POLY = polysomy; IHC = immunohistochemistry; NA = no frozen tissue available; ND = RNA degraded; NT = no tissue slide available
*IHC confirmed by EGFR exon9/exon4 ratio = 4.79

TABLE 3
Biomarkers of response to EGFR kinase inhibitors

| | Responders* | | Non-Responders | | | Odds | 95% |
|---|---|---|---|---|---|---|---|
| UCLA dataset | (n = 7) | | (n = 19) | | P Value | Ratio | C.I. |
| Clinical Variables: | | | | | | | |
| Mean Age | 45 | | 47 | | 0.87 | NA | NA |
| Gender | 4 male/3 female | | 11 male/8 female | | 1 | NA | NA |
| Mean KPS | 88.6 | | 86.3 | | 0.52 | NA | NA |
| Gross total surgical resection | 43% | (3/7) | 42% | (8/19) | 1 | NA | NA |
| EAIED | 29% | (2/7) | 16% | (3/19) | 0.61 | NA | NA |
| EGFR inhibitor | 3 erlotinib/4 geftinib | | 7 erlotinib/12 geftinib | | 1 | NA | NA |
| Mean dose of erlotinib (EIAED−) | 200 mg | (n = 2) | 175 mg | (n = 4) | 0.8 | NA | NA |
| Mean dose of erlotinib (EIAED+) | 500 mg | (n = 1) | 317 mg | (n = 3) | 0.5 | NA | NA |
| Mean dose of geftinib (EIAED−) | 583 mg | (n = 3) | 479 mg | (n = 12) | 0.63 | NA | NA |
| Mean dose of geftinib (EIAED+) | 1500 mg | (n = 1) | (n = 0) | | NA | NA | NA |
| Molecular Biomarkers: | | | | | | | |
| EGFR amplification | 43% | (3/7) | 50% | (6/19) | 0.66 | NA | NA |
| EGFRvIII expression | 83% | (6/7) | 32% | (6/19) | 0.026 | 13 | 1.3-130 |
| PTEN expression | 100% | (7/7) | 32% | (6/19) | 0.0052 | ND** | ND** |
| EGFRvIII/PTEN coexpression**** | 83% | (6/7) | 11% | (2/19) | 0.00078 | 51 | 3.9 = 669 |

| | Responders* | | Non-Responders | | | Odds | 95% |
|---|---|---|---|---|---|---|---|
| UCSF dataset | (n = 6) | | (n = 27) | | P Value | Ratio | C.I. |
| Clinical Variables: | | | | | | | |
| Mean Age | 52 | | 52.7 | | 0.9 | NA | NA |
| Gender | 5 male/1 female | | 11 male/16 female | | 0.09 | NA | NA |
| Concurrent Temozolomide*** | 17% | (1/6) | 19% | (5/27) | 1 | NA | NA |
| EAIED | 83% | (5/6) | 59% | (16/27) | 0.38 | NA | NA |
| Mean dose of erlotinib (EIAED−) | 150 mg | (n = 1) | 182 mg | (n = 11) | 1 | NA | NA |
| Mean dose of erlotinib (EIAED+) | 360 mg | (n = 5) | 281 mg | (n = 16) | 0.39 | NA | NA |
| Molecular Biomarkers: | | | | | | | |
| EGFRvIII expression | 100% | (6/6) | 44% | (12/27) | 0.02 | ND** | ND** |
| PTEN expression | 67% | (4/6) | 19% | (5/27) | 0.034 | 8.8 | 1.3-62 |
| EGFRvIII/PTEN coexpression**** | 67% | (4/6) | 11% | 3/27) | 0.01 | 16 | 2-128 |

Abbreviations: KPS = Karnofsky Performance Score; NA—not applicable; ND—not done
*Response ≧25% tumor shrinkage on MRI in UCLA study; ≧50% tumor shrinkage on MRI in UCSF study
**An odds ratio could not be calculated because none of the UCLA patients with PTEN deficient tumors and none of the UCSF patients lacking EGFRvIII expression responded.
***A subset of patients in the UCSF study received concurrent Temozolomide. All UCLA patients received EGFR inhibitor monotherapy,
****An EGFRvIII/PTEN coexpression molecular diagnostic for clinical response had a sensitivity of 75% and specificity of 94% in the UCLA dataset and a sensitivity of 67% and specificity of 89% in the UCSF dataset

TABLE 4
POLYPEPTIDE SEQUENCES
For convenience, Table 4 provides the se-
quences, accession numbers and illustrative
references for certain well known poly-
peptides discussed herein. In certain sequences
in this Table, illustrative residues that are
typically phosphorylated during pathway
signaling are shown in boldface type.

PTEN (NP 000305, gi: 4506249) 403 amino acids
See, e.g., Li et al., Science 275 (5308),
1943-1947 (1997)
(SEQ ID NO: 1)
MTAIIKEIVSRNKRRYQEDGFDLDLTYIYPNIIAMGFPAERLEGVYRNNI
DDVVRFLDSKHKNHYKIYNLCAERHYDTAKFNCRVAQYPFEDHNPPQLEL
IKPFCEDLDQWLSEDDNHVAAIHCKAGKGRTGVMICAYLLHRGKFLKAQE
ALDFYGEVRTRDKKGVTIPSQRRYVYYYSYLLKNHLDYRPVALLFHKMMF
ETIPMFSGGTCNPQFVVCQLKVKIYSSNSGPTRREDKFMYFEFPQPLPVC
GDIKVEFFHKQNKMLKKDKMFHFWVNTFFIPGPEETSEKVENGSLCDQEI
DSICSIERADNDKEYLVLTLTKNDLDKANKDKANRYFSPNFKVKLYFTKT
VEEPSNPEASSSTSVTPDVSDNEPDHYRYSDTTDSDPENEPFDEDQHTQI
TKV
Epidermal growth factor receptor (erythroblastic
leukemia viral (v-erb-b) oncogene homolog, avian)
(Homo sapiens) (AAS83109, gi 46241840, 1210 amino
acids
(SEQ ID NO: 2)
MRPSGTAGAALLALLAALCPASRALEEKKVCQGTSNKLTQLGTFEDHFLS
LQRMFNNCEVVLGNLEITYVQRNYDLSFLKTIQEVAGYVLIALNTVERIP
LENLQIIRGNMYYENSYALAVLSNYDANKTGLKELPMRNLQEILHGAVRF
SNNPALCNVESIQWRDIVSSDFLSNMSMDFQNHLGSCQKCDPSCPNGSCW
GAGEENCQKLTKIICAQQCSGRCRGKSPSDCCHNQCAAGCTGPRESDCLV
CRKFRDEATCKDTCPPLMLYNPTTYQMDVNPEGKYSFGATCVKKCPRNYV
VTDHGSCVRACGADSYEMEEDGVRKCKKCEGPCRKVCNGIGIGEFKDSLS
INATNIKHFKNCTSISGDLHILPVAFRGDSFTHTPPLDPQELDILKTVKE
ITGFLLIQAWPENRTDLHAFENLEIIRGRTKQHGQFSLAVVSLNITSLGL
RSLKEISDGDVIISGNKNLCYANTINWKKLFGTSGQKTKIISNRGENSCK
ATGQVCHALCSPEGCWGPEPRDCVSCRNVSRGRECVDKCNLLEGEPREFV
ENSECIQCHPECLPQAMNITCTGRGPDNCIQCAHYIDGPHCVKTCPAGVM
GENNTLVWKYADAGHVCHLCHPNCTYGCTGPGLEGCPTNGPKIPSIATGM
VGALLLLLVVALGIGLFMRRRHIVRKRTLRRLLQERELVEPLTPSGEAPN
QALLRILKETEFKKIKVLGSGAFGTVYKGLWIPEGEKVKIPVAIKELREA
TSPKANKEILDEAYVMASVDNPHVCRLLGICLTSTVQLITQLMPFGCLLD
YVREHKDNIGSQYLLNWCVQIAKGMNYLEDRRLVHRDLAARNVLVKTPQH
VKITDFGLAKLLGAEEKEYHAEGGKVPIKWMALESILHRIYTHQSDVWSY
GVTVWELMTFGSKPYDGIPASEISSILEKGERLPQPPICTIDVYMIMVKC
WMIDADSRPKFRELIIEFSKMARDPQRYLVIQGDERMHLPSPTDSNFYRA
LMDEEDMDDVVDADEYLIPQQGFFSSPSTSRTPLLSSLSATSNNSTVACI
DRNGLQSCPIKEDSFLQRYSSDPTGALTEDSIDDTFLPVPEYINQSVPKR
PAGSVQNPVYHNQPLNPAPSRDPHYQDPHSTAVGNPEYLNTVQPTCVNST
FDSPAHWAQKGSHQISLDNPDYQQDFFPKEAKPNGIFKGSTAENAEYLRV
APQSSEFIGA
HER-2/NEU (NP 001005862, gi 54792098) 1225 amino
acids See, e.g., Li M, et al., J of Cancer 118
(4), 801-811 (2006)
(SEQ ID NO: 3)
MKLRLPASPETHLDMLRHLYQGCQVVQGNLELTYLPTNASLSFLQDIQEV
QGYVLIAHNQVRQVPLQRLRIVRGTQLFEDNYALAVLDNGDPLNNTTPVT
GASPGGLRELQLRSLTEILKGGVLIQRNPQLCYQDTILWKDIFHKNNQLA
LTLIDTNRSRACHPCSPMCKGSRCWGESSEDCQSLTRTVCAGGCARCKGP
LPTDCCHEQCAAGCTGPKHSDCLACLHFNHSGICELHCPALVTYNTDTFE
SMPNPEGRYTFGASCVTACPYNYLSTDVGSCTLVCPLHNQEVTAEDGTQR
CEKCSKPCARVCYGLGMEHLREVRAVTSANIQEFAGCKKIFGSLAFLPES
FDGDPASNTAPLQPEQLQVFETLEEITGYLYISAWPDSLPDLSVFQNLQV
IRGRILHNGAYSLTLQGLGISWLGLRSLRELGSGLALIHHNTHLCFVHTV
PWDQLFRNPHQALLHTANRPEDECVGEGLACHQLCARGHCWGPGPTQCVN
CSQFLRGQECVEECRVLQGLPREYVNARHCLPCHPECQPQNGSVTCFGPE
ADQCVACAHYKDPPFCVARCPSGVKPDLSYMPIWKFPDEEGACQPCPINC
THSCVDLDDKGCPAEQRASPLTSIISAVVGILLVVVLGVVFGILIKRRQQ
KIRKYTMRRLLQETELVEPLTPSGAMPNQAQMRILKETELRKVKVLGSGA
FGTVYKGIWIPDGENVKIPVAIKVLRENTSPKANKEILDEAYVMAGVGSP
YVSRLLGICLTSTVQLVTQLMPYGCLLDHVRENRGRLGSQDLLNWCMQIA
KGMSYLEDVRLVHRDLAARNVLVKSPNHVKITDFGLARLLDIDETEYHAD
GGKVPIKWMALESILRRRFTHQSDVWSYGVTVWELMTFGAKPYDGIPARE
IPDLLEKGERLPQPPICTIDVYMIMVKCWMIDSECRPRFRELVSEFSRMA
RDPQRFVVIQNEDLGPASPLDSTFYRSLLEDDDMGDLVDAEEYLVPQQGF
FCPDPAPGAGGMVHHRHRSSSTRSGGGDLTLGLEPSEEEAPRSPLAPSEG
AGSDVFDGDLGMGAAKGLQSLPTHDPSPLQRYSEDPTVPLPSETDGYVAP
LTCSPQPEYVNQPDVRPQPPSPREGPLPAARPAGATLERPKTLSPGKNGV
VKDVFAFGGAVENPEYLTPQGGAAPQPHPPPAFSPAFDNLYYWDQDPPER
GAPPSTFKGTPTAENPEYLGLDVPV
EGFR (NP 005219, gi: 29725609) 1210 amino acids
See, e.g. Tam et al., Nature 309 (5967), 418-425
(1984)
(SEQ ID NO: 4)
MRPSGTAGAALLALLAALCPASRALEEKKVCQGTSNKLTQLGTFEDHFLS
LQRMFNNCEVVLGNLEITYVQRNYDLSFLKTIQEVAGYVLIALNTVERIP
LENLQIIRGNMYYENSYALAVLSNYDANKTGLKELPMRNLQEILHGAVRF
SNNPALCNVESIQWRDIVSSDFLSNMSMDFQNHLGSCQKCDPSCPNGSCW
GAGEENCQKLTKIICAQQCSGRCRGKSPSDCCHNQCAAGCTGPRESDCLV
CRKFRDEATCKDTCPPLMLYNPTTYQMDVNPEGKYSFGATCVKKCPRNYV
VTDHGSCVRACGADSYEMEEDGVRKCKKCEGPCRKVCNGIGIGEFKDSLS
INATNIKHFKNCTSISGDLHILPVAFRGDSFTHTPPLDPQELDILKTVKE
ITGFLLIQAWPENRTDLHAFENLEIIRGRTKQHGQFSLAVVSLNITSLGL
RSLKEISDGDVIISGNKNLCYANTINWKKLFGTSGQKTKIISNRGENSCK
ATGQVCHALCSPEGCWGPEPRDCVSCRNVSRGRECVDKCNLLEGEPREFV
ENSECIQCHPECLPQAMNITCTGRGPDNCIQCAHYIDGPHCVKTCPAGVM
GENNTLVWKYADAGHVCHLCHPNCTYGCTGPGLEGCPTNGPKIPSIATGM
VGALLLLLVVALGIGLFMRRRHIVRKRTLRRLLQERELVEPLTPSGEAPN
QALLRILKETEFKKIKVLGSGAFGTVYKGLWIPEGEKVKIPVAIKELREA
TSPKANKEILDEAYVMASVDNPHVCRLLGICLTSTVQLITQLMPFGCLLD
YVREHKDNIGSQYLLNWCVQIAKGMNYLEDRRLVHRDLAARNVLVKTPQH
VKITDFGLAKLLGAEEKEYHAEGGKVPIKWMALESILHRIYTHQSDVWSY
GVTVWELMTFGSKPYDGIPASEISSILEKGERLPQPPICTIDVYMIMVKC
WMIDADSRPKFRELIIEFSKMARDPQRYLVIQGDERMHLPSPTDSNFYRA
LMDEEDMDDVVDADEYLIPQQGFFSSPSTSRTPLLSSLSATSNNSTVACI
DRNGLQSCPIKEDSFLQRYSSDPTGALTEDSIDDTFLPVPEYINQSVPKR
PAGSVQNPVYHNQPLNPAPSRDPHYQDPHSTAVGNPEYLNTVQPTCVNST
FDSPAHWAQKGSHQISLDNPDYQQDFFPKEAKPNGIFKGSTAENAEYLRV
APQSSEFIGA
S6 (NP 001001, gi: 17158044) 249 amino acids
See, e.g. Pata et al., Gene 121 (2), 387-392
(1992)
(SEQ ID NO: 5)
MKLNISFPATGCQKLIEVDDERKLRTFYEKRMATEVAADALGEEWKGYVV
RISGGNDKQGFPMKQGVLTHGRVRLLLSKGHSCYRPRRTGERKRKSVRGC
IVDANLSVLNLVIVKKGEKDIPGLTDTTVPRRLGPKRASRIRKLFNLSKE
DDVRQYVVRKPLNKEGKKPRTKAPKIQRLVTPRVLQHKRRRIALKKQRTK
KNKEEAAEYAKLLAKRMKEAKEKRQEQIAKRRRLSSLRASTSKSESSQK
AKT (NP 005154 gi: 4885061) 480 amino acids
See, e.g. Staal, S. P., Proc. Natl. Acad. Sci.
U.S.A. 84 (14), 5034-5037 (1987)
(SEQ ID NO: 6)
MSDVAIVKEGWLHKRGEYIKTWRPRYFLLKNDGTFIGYKERPQDVDQREA
PLNNFSVAQCQLMKTERPRPNTFIIRCLQWTTVIERTFHVETPEEREEWT
TAIQTVADGLKKQEEEEMDFRSGSPSDNSGAEEMEVSLAKPKHRVTMNEF
EYLKLLGKGTFGKVILVKEKATGRYYAMKILKKEVIVAKDEVAHTLTENR
VLQNSRHPFLTALKYSFQTHDRLCFVMEYANGGELFFHLSRERVFSEDRA
RFYGAEIVSALDYLHSEKNVVYRDLKLENLMLDKDGHIKITDFGLCKEGI
KDGATMKTFCGTPEYLAPEVLEDNDYGRAVDWWGLGVVMYEMMCGRLPFY
NQDHEKLFELILMEEIRFPRTLGPEAKSLLSGLLKKDPKQRLGGGSEDAK
EIMQHRFFAGIVWQHVYEKKLSPPFKPQVTSETDTRYFDEEFTAQMITIT
PPDQDDSMECVDSERRPHFPQFSYSASSTA
p-ERK (XP 055766, gi: 20562757) 379 amino acids
See, e.g. Butch et al., J Biol Chem. 1996 Feb
23; 271(8): 4230-5.
(SEQ ID NO: 7)
MAAAAAQGGGGGEPRRTEGVGPGVPGEVEMVKGQPFDVGPRYTQLQYIGE
GAYGMVSSAYDHVRKTRVAIKKISPFEHQTYCQRTLREIQILLRFRHENV
IGIRDILRASTLEAMRDVYIVQDLMETDLYKLLKSQQLSNDHICYFLYQI
LRGLKYIHSANVLHRDLKPSNLLINTTCDLKICDFGLARIADPEHDHTGF
LTEYVATRWYRAPEIMLNSKGYTKSIDIWSVGCILAEMLSNRPIFPGKHY
LDQLNHILGILGSPSQEDLNCIINMKARNYLQSLPSKTKVAWAKLFPKSD
SKALDLLDRMLTFNPNKRITVEEALAHPYLEQYYDPTDEPVAEEPFTFAM
ELDDLPKERLKELIFQETARFQPGVLEAP

TABLE 5
POLYNUCLEOTIDE SEQUENCES
For convenience, Table 4 provides the se-
quences, accession numbers and illustrative
references for certain well known poly-
nucleotides discussed herein. In certain
sequences in this Table, illustrative residues
that are typically phosphorylated during
pathway signaling are shown in boldface type.

Homo sapiens phosphatase and tensin homolog
(NM 000314)
(SEQ ID NO: 8)
CCTCCCCTCGCCCGGCGCGGTCCCGTCCGCCTCTCGCTCGCCTCCCGCCT
CCCCTCGGTCTTCCGAGGCGCCCGGGCTCCCGGCGCGGCGGCGGAGGGGG
CGGGCAGGCCGGCGGGCGGTGATGTGGCGGGACTCTTTATGCGCTGCGGC
AGGATACGCGCTCGGCGCTGGGACGCGACTGCGCTCAGTTCTCTCCTCTC
GGAAGCTGCAGCCATGATGGAAGTTTGAGAGTTGAGCCGCTGTGAGGCGA
GGCCGGGCTCAGGCGAGGGAGATGAGAGACGGCGGCGGCCGCGGCCCGGA
GCCCCTCTCAGCGCCTGTGAGCAGCCGCGGGGGCAGCGCCCTCGGGGAGC
CGGCCGGCCTGCGGCGGCGGCAGCGGCGGCGTTTCTCGCCTCCTCTTCGT
CTTTTCTAACCGTGCAGCCTCTTCCTCGGCTTCTCCTGAAAGGGAAGGTG
GAAGCCGTGGGCTCGGGCGGGAGCCGGCTGAGGCGCGGCGGCGGCGGCGG
CACCTCCCGCTCCTGGAGCGGGGGGGAGAAGCGGCGGCGGCGGCGGCCGC
GGCGGCTGCAGCTCCAGGGAGGGGGTCTGAGTCGCCTGTCACCATTTCCA
GGGCTGGGAACGCCGGAGAGTTGGTCTCTCCCCTTCTACTGCCTCCAACA
CGGCGGCGGCGGCGGCGGCACATCCAGGGACCCGGGCCGGTTTTAAACCT
CCCGTCCGCCGCCGCCGCACCCCCCGTGGCCCGGGCTCCGGAGGCCGCCG
GCGGAGGCAGCCGTTCGGAGGATTATTCGTCTTCTCCCCATTCCGCTGCC
GCCGCTGCCAGGCCTCTGGCTGCTGAGGAGAAGCAGGCCCAGTCGCTGCA
ACCATCCAGCAGCCGCCGCAGCAGCCATTACCCGGCTGCGGTCCAGAGCC
AAGCGGCGGCAGAGCGAGGGGCATCAGCTACCGCCAAGTCCAGAGCCATT
TCCATCCTGCAGAAGAAGCCCCGCCACCAGCAGCTTCTGCCATCTCTCTC
CTCCTTTTTCTTCAGCCACAGGCTCCCAGACATGACAGCCATCATCAAAG
AGATCGTTAGCAGAAACAAAAGGAGATATCAAGAGGATGGATTCGACTTA
GACTTGACCTATATTTATCCAAACATTATTGCTATGGGATTTCCTGCAGA
AAGACTTGAAGGCGTATACAGGAACAATATTGATGATGTAGTAAGGTTTT
TGGATTCAAAGCATAAAAACCATTACAAGATATACAATCTTTGTGCTGAA
AGACATTATGACACCGCCAAATTTAATTGCAGAGTTGCACAATATCCTTT
TGAAGACCATAACCCACCACAGCTAGAACTTATCAAACCCTTTTGTGAAG
ATCTTGACCAATGGCTAAGTGAAGATGACAATCATGTTGCAGCAATTCAC
TGTAAAGCTGGAAAGGGACGAACTGGTGTAATGATATGTGCATATTTATT
ACATCGGGGCAAATTTTTAAAGGCACAAGAGGCCCTAGATTTCTATGGGG
AAGTAAGGACCAGAGACAAAAAGGGAGTAACTATTCCCAGTCAGAGGCGC
TATGTGTATTATTATAGCTACCTGTTAAAGAATCATCTGGATTATAGACC
AGTGGCACTGTTGTTTCACAAGATGATGTTTGAAACTATTCCAATGTTCA
GTGGCGGAACTTATCCTCAGTTTGTGGTCTGCCAGCTAAAGGTGAAGATA
TATTCCTCCAATTCAGGACCCACACGACGGGAAGACAAGTTCATGTACTT
TGAGTTCCCTCAGCCGTTACCTGTGTGTGGTGATATCAAAGTAGAGTTCT
TCCACAAACAGAACAAGATGCTAAAAAAGGACAAAATGTTTCACTTTTGG
GTAAATACATTCTTCATACCAGGACCAGAGGAAACCTCAGAAAAAGTAGA
AAATGGAAGTCTATGTGATCAAGAAATCGATAGCATTTGCAGTATAGAGC
GTGCAGATAATGACAAGGAATATCTAGTACTTACTTTAACAAAAAATGAT
CTTGACAAAGCAAATAAAGACAAAGCCAACCGATACTTTTCTCCAAATTT
TAAGGTGAAGCTGTACTTCACAAAAACAGTAGAGGAGCCGTCAAATCCAG
AGGCTAGCAGTTCAACTTCTGTAACACCAGATGTTAGTGACAATGAACCT
GATCATTATAGATATTCTGACACCACTGACTCTGATCCAGAGAATGAACC
TTTTGATGAAGATCAGCATACACAAATTACAAAAGTCTGAATTTTTTTTT
ATCAAGAGGGATAAAACACCATGAAAATAAACTTGAATAAACTGAAAATG
GACCTTTTTTTTTTTAATGGCAATAGGACATTGTGTCAGATTACCAGTTA
TAGGAACAATTCTCTTTTCCTGACCAATCTTGTTTTACCCTATACATCCA
CAGGGTTTTGACACTTGTTGTCCAGTTGAAAAAAGGTTGTGTAGCTGTGT
CATGTATATACCTTTTTGTGTCAAAAGGACATTTAAAATTCAATTAGGAT
TAATAAAGATGGCACTTTCCCGTTTTATTCCAGTTTTATAAAAAGTGGAG
ACAGACTGATGTGTATACGTAGGAATTTTTTCCTTTTGTGTTCTGTCACC
AACTGAAGTGGCTAAAGAGCTTTGTGATATACTGGTTCACATCCTACCCC
TTTGCACTTGTGGCAACAGATAAGTTTGCAGTTGGCTAAGAGAGGTTTCC
GAAGGGTTTTGCTACATTCTAATGCATGTATTCGGGTTAGGGGAATGGAG
GGAATGCTCAGAAAGGAAATAATTTTATGCTGGACTCTGGACCATATACC
ATCTCCAGCTATTTACACACACCTTTCTTTAGCATGCTACAGTTATTAAT
CTGGACATTCGAGGAATTGGCCGCTGTCACTGCTTGTTGTTTGCGCATTT
TTTTTTAAAGCATATTGGTGCTAGAAAAGGCAGCTAAAGGAAGTGAATCT
GTATTGGGGTACAGGAATGAACCTTCTGCAACATCTTAAGATCCACAAAT
GAAGGGATATAAAAATAATGTCATAGGTAAGAAACACAGCAACAATGACT
TAACCATATAAATGTGGAGGCTATCAACAAAGAATGGGCTTGAAACATTA
TAAAAATTGACAATGATTTATTAAATATGTTTTCTCAATTGTAACGACTT
CTCCATCTCCTGTGTAATCAAGGCCAGTGCTAAAATTCAGATGCTGTTAG
TACCTACATCAGTCAACAACTTACACTTATTTTACTAGTTTTCAATCATA
ATACCTGCTGTGGATGCTTCATGTGCTGCCTGCAAGCTTCTTTTTTCTCA
TTAAATATAAAATATTTTGTAATGCTGCACAGAAATTTTCAATTTGAGAT
TCTACAGTAAGCGTTTTTTTTCTTTGAAGATTTATGATGCACTTATTCAA
TAGCTGTCAGCCG
Homo sapiens epidermal growth factor receptor
(erythroblastic leukemia viral (v-erb-b) oncogene
homolog, avian) (EGFR), transcript variant 3, cDNA
(NM 201283)
(SEQ ID NO: 9)
CCCCGGCGCAGCGCGGCCGCAGCAGCCTCCGCCCCCCGCACGGTGTGAGC
GCCCGACGCGGCCGAGGCGGCCGGAGTCCCGAGCTAGCCCCGGCGGCCGC
CGCCGCCCAGACCGGACGACAGGCCACCTCGTCGGCGTCCGCCCGAGTCC
CCGCCTCGCCGCCAACGCCACAACCACCGCGCACGGCCCCCTGACTCCGT
CCAGTATTGATCGGGAGAGCCGGAGCGAGCTCTTCGGGGAGCAGCGATGC
GACCCTCCGGGACGGCCGGGGCAGCGCTCCTGGCGCTGCTGGCTGCGCTC
TGCCCGGCGAGTCGGGCTCTGGAGGAAAAGAAAGTTTGCCAAGGCACGAG
TAACAAGCTCACGCAGTTGGGCACTTTTGAAGATCATTTTCTCAGCCTCC
AGAGGATGTTCAATAACTGTGAGGTGGTCCTTGGGAATTTGGAAATTACC
TATGTGCAGAGGAATTATGATCTTTCCTTCTTAAAGACCATCCAGGAGGT
GGCTGGTTATGTCCTCATTGCCCTCAACACAGTGGAGCGAATTCCTTTGG
AAAACCTGCAGATCATCAGAGGAAATATGTACTACGAAAATTCCTATGCC
TTAGCAGTCTTATCTAACTATGATGCAAATAAAACCGGACTGAAGGAGCT
GCCCATGAGAAATTTACAGGAAATCCTGCATGGCGCCGTGCGGTTCAGCA
ACAACCCTGCCCTGTGCAACGTGGAGAGCATCCAGTGGCGGGACATAGTC
AGCAGTGACTTTCTCAGCAACATGTCGATGGACTTCCAGAACCACCTGGG
CAGCTGCCAAAAGTGTGATCCAAGCTGTCCCAATGGGAGCTGCTGGGGTG
CAGGAGAGGAGAACTGCCAGAAACTGACCAAAATCATCTGTGCCCAGCAG
TGCTCCGGGCGCTGCCGTGGCAAGTCCCCCAGTGACTGCTGCCACAACCA
GTGTGCTGCAGGCTGCACAGGCCCCCGGGAGAGCGACTGCCTGGTCTGCC
GCAAATTCCGAGACGAAGCCACGTGCAAGGACACCTGCCCCCCACTCATG
CTCTACAACCCCACCACGTACCAGATGGATGTGAACCCCGAGGGCAAATA
CAGCTTTGGTGCCACCTGCGTGAAGAAGTGTCCCCGTAATTATGTGGTGA
CAGATCACGGCTCGTGCGTCCGAGCCTGTGGGGCCGACAGCTATGAGATG
GAGGAAGACGGCGTCCGCAAGTGTAAGAAGTGCGAAGGGCCTTGCCGCAA
AGTGTGTAACGGAATAGGTATTGGTGAATTTAAAGACTCACTCTCCATAA
ATGCTACGAATATTAAACACTTCAAAAACTGCACCTCCATCAGTGGCGAT
CTCCACATCCTGCCGGTGGCATTTAGGGGTGACTCCTTCACACATACTCC
TCCTCTGGATCCACAGGAACTGGATATTCTGAAAACCGTAAAGGAAATCA
CAGGTTTGAGCTGAATTATCACATGAATATAAATGGGAAATCAGTGTTTT
AGAGAGAGAACTTTTCGACATATTTCCTGTTCCCTTGGAATAAAAACATT
TCTTCTGAAATTTTACCGTTAAAAAAAAAAAAAAAAAAAAAAAAA

What is claimed is:

1. A method for identifying a mammalian tumor cell that is likely to respond, or is responsive to an epidermal growth factor receptor (EGFR) inhibitor, the method comprising examining the cell for the expression of EGFR deletion mutant variant III (“EGFRvIII”, SEQ ID NO: 2) and the expression of phosphatase and tensin homologue deleted on chromosome 10 (“PTEN”, SEQ ID NO: 1), wherein the coexpression of EGFRvIII and PTEN identifies the cell as likely to respond or responsive to an epidermal growth factor receptor (EGFR) inhibitor.

2. The method of claim 1, wherein the mammalian tumor cell is a glioma.

3. The method of claim 1, wherein the coexpression of EGFRvIII and PTEN is examined using an antibody that binds EGFRvIII protein and an antibody that binds PTEN protein.

4. The method of claim 3, wherein the coexpression of EGFRvIII and PTEN is examined using immunohistochemistry or immunoblotting.

5. The method of claim 1, wherein the coexpression of EGFRvIII and PTEN are evaluated by contacting the cell with a polynucleotide that hybridizes to EGFRvIII polynucleotide (SEQ ID NO: 9) and a polynucleotide that hybridizes to PTEN polynucleotide (SEQ ID NO: 8).

6. The method of claim 5, wherein the coexpression of EGFRvIII and PTEN in the cell are evaluated by Northern analysis or polymerase chain reaction analysis.

7. The method of claim 1, wherein the cell does not express EGFR (SEQ ID NO: 4) having a deletion mutation in a kinase domain.

8. The method of claim 1, wherein the cell does not express HER2 (SEQ ID NO: 3) having a deletion mutation in a kinase domain.

9. The method of claim 1, wherein the cell does not exhibit amplification of the gene that encodes EGFR (SEQ ID NO: 4).

10. The method of claim 1, wherein the epidermal growth factor receptor (EGFR) inhibitor comprises erlotinib, gefitinib, ZD-1839, OSI-774, PD-153053, PD-168393, IMC-C225, CI-1033, AG1478, 4-(2′fluoroanilino)-and 4-(3′fluoroanilino)-6,7 diethoxyquinazoline, 4-(3′bromoanilino-6,7-ditnethoxyquinazoline), AX7593, PP2, pyrrole(2,1-f)(1,2,4) triazine nucleus, 5-substituted-4-hydroxy-8-nitroquinazoline, EKB-569, MSK-039, cetuximab, benzamide, benzamidine, acryloylamino-salicylanilides, or HKI-272.

11. The method of claim 1, wherein the mammalian tumor cell exhibits disregulation of the PI3K/AKT pathway.

12. A method for identifying a mammalian tumor cell that is likely to respond, or is responsive to an epidermal growth factor receptor (EGFR) inhibitor, the method comprising examining the cell for the expression of EGFR deletion mutant variant III polypeptide (“EGFRvIII”, SEQ ID NO: 2) and the expression of phosphatase and tensin homologue deleted on chromosome 10 polypeptide (“PTEN”, SEQ ID NO: 1) using an antibody that binds EGFRvIII polypeptide and an antibody that binds PTEN polypeptide, wherein:
the mammalian tumor cell is in a paraffin embedded tissue section derived from a patient biopsy; and
the coexpression of EGFRvIII polypeptide and PTEN polypeptide identifies the cell as likely to respond or responsive to an epidermal growth factor receptor (EGFR) inhibitor.

13. The method of claim 12, further comprising examining the cell for phosphorylated S6 ribosomal polypeptide (SEQ ID NO: 5); phosphorylated AKT polypeptide (SEQ ID NO: 6); or phosphorylated ERK polypeptide (SEQ ID NO: 7)

14. The method of claim 13, wherein the presence of phosphorylated S6 ribosomal polypeptide (SEQ ID NO: 5) is examined using an antibody that binds an epitope comprising a phosphorylated serine residue at position 235 in SEQ ID NO: 5; the presence of phosphorylated AKT (SEQ ID NO: 6) is examined using an antibody that binds an epitope comprising a phosphorylated serine residue at position 473 in SEQ ID NO: 6; and the presence of phosphorylated ERK is examined using an antibody that binds an epitope comprising a phosphorylated threonine residue at position 202 or a phosphorylated tyrosine residue at position 204 in SEQ ID NO: 7.

15. The method of claim 12, wherein the mammalian tumor cell is a glioma.

16. The method of claim 15, wherein the tumor is a glioblastoma multiforme tumor.

17. The method of claim 12, wherein the expression of EGFR deletion mutant variant III polypeptide and the expression of phosphatase and tensin homologue deleted on chromosome 10 polypeptide is determined subsequent to contacting the cell with an EGFR inhibitor.

18. The method of claim 12, wherein the epidermal growth factor receptor (EGFR) inhibitor is comprises erlotinib, gefitinib, ZD-1839, OSI-774, PD-153053, PD-168393, IMC-C225, CI-1033, AG1478, 4-(2′fluoro anilino)-and 4-(3′fluoroanilino)-6,7 diethoxyquinazoline, 4-(3′bromoanilino-6,7-dimethoxyquinazoline), AX7593, PP2, pyrrole(2,1-f)(1,2,4) triazine nucleus, 5-substituted-4-hydroxy-8-nitroquinazoline, EKB-569, MSK-039, cetuximab, benzamide, benzamidine, acryloylamino-salicylanilides, or HKI-272.

19. A kit for characterizing a mammalian tumor or cell, the kit comprising:
(a) an antibody that binds EGFR deletion mutant variant III polypeptide (“EGFRvIII”, SEQ ID NO: 2);
(b) an antibody that binds phosphatase and tensin homologue deleted on chromosome 10 polypeptide (“PTEN”, SEQ ID NO: 1);
(c) a container for (a) and (b); and
(d) instructions for using the kit.

20. The kit of claim 19, wherein the kit further includes; and at least one secondary antibody that binds to an antibody (a) or (b).

21. A kit for characterizing a mammalian tumor or cell, the kit comprising:
(a) a polynucleotide that hybridizes to EGFR deletion mutant variant III polynucleotide (SEQ ID NO: 9);
(b) a polynucleotide that hybridizes to phosphatase and tensin homologue deleted on chromosome 10 polynucleotide (SEQ ID NO: 8);
(c) a container for (a) and (b); and
(d) instructions for using the kit.
